(19) United States
(12) Patent Application Publication
(10) Pub. No.: US 2023/0310436 A1
(43) Pub. Date: Oct. 5, 2023

(54) COMBINATION THERAPY FOR CANCERS WITH KRAS MUTATION

(71) Applicant: COTHERA BIOSCIENCE, INC., Grand Cayman (KY)

(72) Inventors: Yiyou CHEN, Beijing (CN); Chun JIANG, Hillsborough, CA (US)

(73) Assignee: COTHERA BIOSCIENCE, INC., Grand Cayman (KY)

(21) Appl. No.: 17/766,724

(22) PCT Filed: Oct. 8, 2020
(86) PCT No.: PCT/CN2020/119873

§ 371 Date: Apr. 5, 2022

(30) Foreign Application Priority Data
Oct. 9, 2019 (WO) PCT/CN2019/110112

(51) Int. Cl. A61K 31/519; A61P 35/00; A61K 31/506; A61K 31/4523; A61K 31/517; A61K 39/395

(52) U.S. Cl. CPC A61K 31/519; A61P 35/00; A61K 31/506; A61K 31/4523; A61K 31/517; A61K 39/3955; A61K 2039/505

(57) ABSTRACT

Provided is a combination therapy for treating cancer with KRAS mutations comprising administrating to a subject an effective amount of (a) an epidermal growth factor receptor (EGFR) inhibitor, (b) a mitogen-activated protein kinase (MEK) 1/2 inhibitor, and (c) a cyclin dependent kinase (CDK) 4/6 inhibitor. Also provided are compositions and kits related to the combination therapy.

### CROSS REFERENCE TO RELATED APPLICATION

This application claims the benefit of International Application No. PCT/CN2019/110112, filed Oct. 9, 2019, which is hereby incorporated by reference in its entirety.

### FIELD OF INVENTION

The present disclosure relates generally to combination therapies for treating or delaying progression of cancers with a KRAS mutation and related compositions and kits.

### BACKGROUND

The KRAS is part of the RAS/MAPK signaling pathway, which is involved in the regulation of cell proliferation, survival and differentiation. The KRAS protein is a GTPase which cycles between inactive guanosine diphosphate (GDP)-bound and active guanosine triphosphate (GTP)-bound forms. KRAS has been implicated in the pathogenesis of several cancers, including but not limited to, lung cancer, colorectal cancer and pancreatic cancer. Lung cancer is the most common cancer worldwide and the second most common cancer in the US with estimated death of ˜154,000 in 2018. See seer.cancer.gov/statfacts/html/lungb.html. Non-small cell lung cancer (NSCLC) accounts for about 85% of all lung cancers and KRAS is implicated in about 22% of NSCLC. See Mayo Clin Proc. 2008 May; 83(5): 584-594 and EBioMedicine 2019 March; 41: 711-716. Nearly all (˜97%) of the KRAS gene mutations associated with lung cancer change the amino acid glycine at position 12 or 13 (Gly12 or Gly13) or change the amino acid glutamine at position 61 (Gln61) in the K-Ras protein. See Proc Am Thorac Soc 2009 April; 6: 201-205. G12C, G12D and G12V are among the major KRAS mutations found in NSCLC, accounting for ˜43%, ˜11% and ˜18%, respectively, and are highly indicative of this form of lung cancer. See EBioMedicine 2019 March; 41: 711-716. There is currently no approved target therapy to treat NSCLC harboring KRAS mutations (KRAS NSCLC). See Mol Cancer 2018 February; 17: 33 and Lung Cancer, 2018 October, 124: 53-64.

Current KRAS inhibitors mainly target the KRAS G12C mutations (see, e.g. Nature Reviews Drug Discovery 18, 887-891 (2019)). However, KRAS G12C only account for a small portion of all possible KRAS mutations in cancer patients. See, Nat Rev Drug Discov. 2014; 13(11):828-851. Therefore, there remains a need for a robust pan-KRAS therapy for treating cancer (e.g., lung cancer) with KRAS mutations.

The disclosures of all publications, patents, patent applications and published patent applications referred to herein are hereby incorporated herein by reference in their entirety.

### BRIEF SUMMARY

Provided herein are compositions comprising (a) an epidermal growth factor receptor (EGFR) inhibitor; (b) a mitogen-activated protein kinase (MEK) 1/2 inhibitor; and (c) a cyclin dependent kinase (CDK) 4/6 inhibitor; wherein the composition does not comprises a KRAS inhibitor. In some embodiments, the composition comprises osimertinib or a salt thereof, cobimetinib or a salt thereof and palbociclib or a salt thereof, wherein the composition does not comprises a KRAS inhibitor. In some embodiments, the composition comprises osimertinib or a salt thereof, TAK-733 or a salt thereof and palbociclib or a salt thereof, wherein the composition does not comprises a KRAS inhibitor. In some embodiments, the composition comprises cetuximab, TAK-733 or a salt thereof and palbociclib or a salt thereof, wherein the composition does not comprises a KRAS inhibitor. In some embodiments, the composition comprises cetuximab, cobimetinib or a salt thereof and palbociclib or a salt thereof, wherein the composition does not comprises a KRAS inhibitor. In some embodiments, the composition comprises avitinib or a salt thereof, cobimetinib or a salt thereof, and palbociclib or a salt thereof, wherein the composition does not comprises a KRAS inhibitor. In some embodiments, the composition comprises osimertinib or a salt thereof, binimetinib or a salt thereof, and palbociclib or a salt thereof, wherein the composition does not comprises a KRAS inhibitor. In some embodiments, the composition comprises cetuximab, binimetinib or a salt thereof, and palbociclib or a salt thereof, wherein the composition does not comprises a KRAS inhibitor. In some embodiments, the composition comprises cetuximab, cobimetinib or a salt thereof, and abemaciclib or a salt thereof, wherein the composition does not comprises a KRAS inhibitor. In some embodiments, the composition consists of osimertinib or a salt thereof, cobimetinib or a salt thereof and palbociclib or a salt thereof. In some embodiments, the composition consists of cetuximab, cobimetinib or a salt thereof and palbociclib or a salt thereof. In some embodiments, the composition consists of osimertinib or a salt thereof, TAK-733 or a salt thereof and palbociclib or a salt thereof. In some embodiments, the composition consists of cetuximab, TAK-733 or a salt thereof and palbociclib or a salt thereof. In some embodiments, the composition consists of avitinib or a salt thereof, cobimetinib or a salt thereof, and palbociclib or a salt thereof. In some embodiments, the composition consists of osimertinib or a salt thereof, binimetinib or a salt thereof, and palbociclib or a salt thereof. In some embodiments, the composition consists of cetuximab, binimetinib or a salt thereof, and palbociclib or a salt thereof. In some embodiments, the composition consists of cetuximab, cobimetinib or a salt thereof, and abemaciclib or a salt thereof. In some embodiments, the composition further comprises a pharmaceutically acceptable carrier, excipient, binder, or diluent. In some embodiments, the composition is formulated for oral administration to a subject.

Also provided here are methods for treating or delaying progression of cancer (e.g., lung cancer and pancreatic cancer) in a subject comprising administering to the subject an effective amount of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor, wherein the subject has cancer that has a KRAS mutation or is at risk of developing cancer that has a KRAS mutation. In some embodiments, the method does not comprise administering a KRAS inhibitor to the subject during the treatment. In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the cancer is a lung cancer. In some embodiments, the lung cancer is non-small cell lung cancer (NSCLC).

Also provided here are methods for treating or delaying progression of cancer in a subject comprising administering to the subject an effective amount of osimertinib or a salt thereof, cobimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, cobimetinib or a salt thereof, and palbociclib or a salt thereof. In some embodiments, the method does not comprise administering to the subject an additional therapeutic agent during the administrations of osimertinib or a salt thereof, cobimetinib or a salt thereof, and palbociclib or a salt thereof. In some embodiments, osimertinib or a salt thereof, cobimetinib or a salt thereof, and palbociclib or a salt thereof are administered in one composition. In some embodiments, cobimetinib or a salt thereof, and palbociclib or a salt thereof are administered in two or more compositions (e.g., two or three compositions). In some embodiments, osimertinib or a salt thereof, cobimetinib or a salt thereof, and palbociclib or a salt thereof are administered continuously to the subject. In some embodiments, osimertinib or a salt thereof, cobimetinib or a salt thereof, and palbociclib or a salt thereof are administered intermittently to the subject. In some embodiments, the cancer is a lung cancer. In some embodiments, the lung cancer is NSCLC.

In some embodiments, osimertinib or a salt thereof is administered to the subject in a daily dose of about 40-160 mg. In some embodiments, cobimetinib or a salt thereof is administered to the subject in a daily dose of about 20-60 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, cobimetinib or a salt thereof is administered to the subject in a daily dose of about 0.25-1 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, cobimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, cobimetinib or a salt thereof, and palbociclib or a salt thereof. In some embodiments, the method does not comprise administering to the subject an additional therapeutic agent during the administrations of cetuximab, cobimetinib or a salt thereof, and palbociclib or a salt thereof. In some embodiments, cetuximab, cobimetinib or a salt thereof, and palbociclib or a salt thereof are administered in one composition. In some embodiments, cetuximab, cobimetinib or a salt thereof, and palbociclib or a salt thereof are administered in two or more compositions (e.g., two or three compositions). In some embodiments, cetuximab, cobimetinib or a salt thereof, and palbociclib or a salt thereof are administered continuously to the subject. In some embodiments, cetuximab, cobimetinib or a salt thereof, and palbociclib or a salt thereof are administered intermittently to the subject. In some embodiments, the cancer is a lung cancer. In some embodiments, the lung cancer is NSCLC.

In some embodiments, cetuximab is administered to the subject in 400 mg/m2 infused over 120 minutes followed by 250 mg/m² weekly infused over 60 minutes. In some embodiments, the maximum infusion rate is about 10 mL/min. In some embodiments, cobimetinib or a salt thereof is administered to the subject in a daily dose of about 20-60 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, cobimetinib or a salt thereof is administered to the subject in a daily dose of about 0.25-10 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, TAK-733 or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, TAK-733 or a salt thereof, and palbociclib or a salt thereof. In some embodiments, the method does not comprise administering to the subject an additional therapeutic agent during the administrations of cetuximab, TAK-733 or a salt thereof, and palbociclib or a salt thereof. In some embodiments, cetuximab, TAK-733 or a salt thereof, and palbociclib or a salt thereof are administered in one composition. In some embodiments, cetuximab, TAK-733 or a salt thereof and palbociclib or a salt thereof are administered in two or more compositions (e.g., two or three compositions). In some embodiments, cetuximab, TAK-733 or a salt thereof, and palbociclib or a salt thereof are administered continuously to the subject. In some embodiments, cetuximab, TAK-733 or a salt thereof, and palbociclib or a salt thereof are administered intermittently to the subject. In some embodiments, the cancer is a lung cancer. In some embodiments, the lung cancer is NSCLC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, TAK-733 or a salt thereof or a salt thereof is administered to the subject in a daily dose of about 20-60 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, TAK-733 or a salt thereof is administered to the subject in a daily dose of about 0.25-30 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, TAK-733 or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, TAK-733 or a salt thereof, and palbociclib or a salt thereof. In some embodiments, the method does not comprise administering to the subject an additional therapeutic agent during the administrations of osimertinib or a salt thereof, TAK-733 or a salt thereof, and palbociclib or a salt thereof. In some embodiments, osimertinib or a salt thereof, TAK-733 or a salt thereof, and palbociclib or a salt thereof are administered in one composition. In some embodiments, osimertinib or a salt thereof, TAK-733 or a salt thereof and palbociclib or a salt thereof are administered in two or more compositions (e.g., two or three compositions). In some embodiments, osimertinib or a salt thereof, TAK-733 or a salt thereof, and palbociclib or a salt thereof are administered continuously to the subject. In some embodiments, osimertinib or a salt thereof, TAK-733 or a salt thereof, and palbociclib or a salt thereof are administered intermittently to the subject. In some embodiments, the cancer is a lung cancer. In some embodiments, the lung cancer is NSCLC.

In some embodiments, osimertinib or a salt thereof is administered to the subject in a daily dose of about 40-160 mg. In some embodiments, TAK-733 or a salt thereof or a salt thereof is administered to the subject in a daily dose of about 20-60 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, TAK-733 or a salt thereof is administered to the subject in a daily dose of about 0.25-30 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of avitinib or a salt thereof, cobimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of avitinib or a salt thereof, cobimetinib or a salt thereof, and palbociclib or a salt thereof. In some embodiments, the method does not comprise administering to the subject an additional therapeutic agent during the administrations of avitinib or a salt thereof, cobimetinib or a salt thereof, and palbociclib or a salt thereof. In some embodiments, avitinib or a salt thereof, cobimetinib or a salt thereof, and palbociclib or a salt thereof are administered in one composition. In some embodiments, avitinib or a salt thereof, cobimetinib or a salt thereof, and palbociclib or a salt thereof are administered in two or more compositions (e.g., two or three compositions). In some embodiments, avitinib or a salt thereof, cobimetinib or a salt thereof, and palbociclib or a salt thereof are administered continuously to the subject. In some embodiments, avitinib or a salt thereof, cobimetinib or a salt thereof, and palbociclib or a salt thereof are administered intermittently to the subject. In some embodiments, the cancer is a lung cancer. In some embodiments, the lung cancer is NSCLC.

In some embodiments, avitinib or a salt thereof is administered to the subject in a daily dose of about 40-160 mg. In some embodiments, cobimetinib or a salt thereof or a salt thereof is administered to the subject in a daily dose of about 20-60 mg. In some embodiments, palbociclib or a salt thereof is administered to the subject in a daily dose of about 15-125 mg. In some embodiments, avitinib or a salt thereof is administered to the subject in a daily dose of about 0.5-3 mg/kg. In some embodiments, cobimetinib or a salt thereof is administered to the subject in a daily dose of about 0.25-30 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, binimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, binimetinib or a salt thereof, and palbociclib or a salt thereof. In some embodiments, the method does not comprise administering to the subject an additional therapeutic agent during the administrations of osimertinib or a salt thereof, binimetinib or a salt thereof, and palbociclib or a salt thereof. In some embodiments, osimertinib or a salt thereof, binimetinib or a salt thereof, and palbociclib or a salt thereof are administered in one composition. In some embodiments, osimertinib or a salt thereof, binimetinib or a salt thereof and palbociclib or a salt thereof are administered in two or more compositions (e.g., two or three compositions). In some embodiments, osimertinib or a salt thereof, binimetinib or a salt thereof, and palbociclib or a salt thereof are administered continuously to the subject. In some embodiments, osimertinib or a salt thereof, binimetinib or a salt thereof, and palbociclib or a salt thereof are administered intermittently to the subject. In some embodiments, the cancer is a lung cancer. In some embodiments, the lung cancer is NSCLC.

In some embodiments, osimertinib or a salt thereof is administered to the subject in a daily dose of about 40-160 mg. In some embodiments, binimetinib or a salt thereof or a salt thereof is administered to the subject in a daily dose of about 60-90 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, binimetinib or a salt thereof is administered to the subject in a daily dose of about 0.8-2.7 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, binimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, binimetinib or a salt thereof, and palbociclib or a salt thereof. In some embodiments, the method does not comprise administering to the subject an additional therapeutic agent during the administrations of cetuximab, binimetinib or a salt thereof, and palbociclib or a salt thereof. In some embodiments, cetuximab, binimetinib or a salt thereof, and palbociclib or a salt thereof are administered in one composition. In some embodiments, cetuximab, binimetinib or a salt thereof and palbociclib or a salt thereof are administered in two or more compositions (e.g., two or three compositions). In some embodiments, cetuximab, binimetinib or a salt thereof, and palbociclib or a salt thereof are administered continuously to the subject. In some embodiments, cetuximab, binimetinib or a salt thereof, and palbociclib or a salt thereof are administered intermittently to the subject. In some embodiments, the cancer is a lung cancer. In some embodiments, the lung cancer is NSCLC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, binimetinib or a salt thereof or a salt thereof is administered to the subject in a daily dose of about 60-90 mg. In some embodiments, palbociclib or a salt thereof is administered to the subject in a daily dose of about 15-125 mg. In some embodiments, cetuximab is administered to the subject in a daily dose of about 0.5-3 mg/kg. In some embodiments, binimetinib or a salt thereof is administered to the subject in a daily dose of about 0.8-2.7 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, trametinib or a salt thereof and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, trametinib or a salt thereof and palbociclib or a salt thereof. In some embodiments, the method does not comprise administering to the subject an additional therapeutic agent during the administrations of cetuximab, trametinib or a salt thereof, and palbociclib or a salt thereof. In some embodiments, cetuximab, trametinib or a salt thereof, and palbociclib or a salt thereof are administered in one composition. In some embodiments, cetuximab, trametinib or a salt thereof and palbociclib or a salt thereof are administered in two or more compositions (e.g., two or three compositions). In some embodiments, cetuximab, trametinib or a salt thereof, and palbociclib or a salt thereof are administered continuously to the subject. In some embodiments, cetuximab, trametinib or a salt thereof, and palbociclib or a salt thereof are administered intermittently to the subject. In some embodiments, the cancer is a lung cancer. In some embodiments, the lung cancer is NSCLC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, trametinib or a salt thereof or a salt thereof is administered to the subject in a daily dose of about 2 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, trametinib or a salt thereof is administered to the subject in a daily dose of about 0.8-2.7 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, trametinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, trametinib or a salt thereof, and palbociclib or a salt thereof. In some embodiments, the method does not comprise administering to the subject an additional therapeutic agent during the administrations of osimertinib or a salt thereof, trametinib or a salt thereof, and palbociclib or a salt thereof. In some embodiments, osimertinib or a salt thereof, trametinib or a salt thereof, and palbociclib or a salt thereof are administered in one composition. In some embodiments, trametinib or a salt thereof, and palbociclib or a salt thereof are administered in two or more compositions (e.g., two or three compositions). In some embodiments, osimertinib or a salt thereof, trametinib or a salt thereof, and palbociclib or a salt thereof are administered continuously to the subject. In some embodiments, osimertinib or a salt thereof, trametinib or a salt thereof, and palbociclib or a salt thereof are administered intermittently to the subject. In some embodiments, the cancer is a lung cancer. In some embodiments, the lung cancer is NSCLC.

In some embodiments, osimertinib or a salt thereof is administered to the subject in a daily dose of about 40-160 mg. In some embodiments, trametinib or a salt thereof is administered to the subject in a daily dose of about 0.5-2 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, trametinib or a salt thereof is administered to the subject in a daily dose of about 0.25-1 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, cobimetinib or a salt thereof, and abemaciclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, cobimetinib or a salt thereof, and abemaciclib or a salt thereof. In some embodiments, the method does not comprise administering to the subject an additional therapeutic agent during the administrations of cetuximab, cobimetinib or a salt thereof, and abemaciclib or a salt thereof. In some embodiments, cetuximab, cobimetinib or a salt thereof, and abemaciclib or a salt thereof are administered in one composition. In some embodiments, cetuximab, cobimetinib or a salt thereof and abemaciclib or a salt thereof are administered in two or more compositions (e.g., two or three compositions). In some embodiments, cetuximab, cobimetinib or a salt thereof, and abemaciclib or a salt thereof are administered continuously to the subject. In some embodiments, cetuximab, cobimetinib or a salt thereof, and abemaciclib or a salt thereof are administered intermittently to the subject. In some embodiments, the cancer is a lung cancer. In some embodiments, the lung cancer is NSCLC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, cobimetinib or a salt thereof or a salt thereof is administered to the subject in a daily dose of about 60-90 mg. In some embodiments, abemaciclib or a salt thereof is administered to the subject in a daily dose of about 100-400 mg. In some embodiments, cetuximab is administered to the subject in a daily dose of about 0.5-3 mg/kg. In some embodiments, cobimetinib or a salt thereof is administered to the subject in a daily dose of about 0.8-2.7 mg/kg. In some embodiments, abemaciclib or a salt thereof is administered to the subject in a daily dose of about 0.25-2.5 mg/kg

In some embodiments, the cancer has a KRAS G12 mutation or a KRAS G13 mutation. In some embodiments, the KRAS G12 mutation is KRAS G12C, G12D or G12V mutation. In some embodiments, the KRAS G12 mutation is KRAS G12C or G12V mutation. In some embodiments, the cancer is a malignant epithelial tumor or carcinoma. In some embodiments, the cancer is a carcinoma selected from one or more of a lung cancer, colorectal cancer and pancreatic cancer. In some embodiments, the cancer is a lung cancer. In some embodiments, the lung cancer is NSCLC. In some embodiments, the NSCLC has a KRAS G12C, G12D, or G12V mutation. In some embodiments, the NSCLC has a KRAS G13D mutation.

In some embodiments, the subject had received a KRAS inhibitor in a previous treatment cycle. In some embodiments, the subject has not received a KRAS inhibitor in a previous treatment cycle.

In some embodiments, the method provided herein reduces cancer cell growth and/or increase cancer cell-killing by about 20-99% more than administration of (a) an epidermal growth factor receptor (EGFR) inhibitor; (b) a mitogen-activated protein kinase (MEK) 1/2 inhibitor; or (c) a cyclin dependent kinase (CDK) 4/6 inhibitor alone. In some embodiments, the method provided herein reduces cancer cell growth and/or increase cancer cell-killing by about 20-99% more than administration of osimertinib or a salt thereof, cobimetinib or a salt thereof, or palbociclib or a salt thereof alone. In some embodiments, the method provided herein reduces cancer cell growth and/or increase cancer cell-killing by about 20-99% more than administration of cetuximab, cobimetinib or a salt thereof, or palbociclib or a salt thereof alone. In some embodiments, the method provided herein reduces cancer cell growth and/or increase cancer cell-killing by about 20-99% more than administration of cetuximab, TAK-733 or a salt thereof, or palbociclib or a salt thereof alone. In some embodiments, the method provided herein reduces cancer cell growth and/or increase cancer cell-killing by about 20-99% more than administration of osimertinib or a salt thereof, TAK-733 or a salt thereof, or palbociclib or a salt thereof alone. In some embodiments, the method reduces tumor volume by about 20-95%.

In another aspect, provided herein are kits comprising (a) an epidermal growth factor receptor (EGFR) inhibitor; (b) a mitogen-activated protein kinase (MEK) 1/2 inhibitor; and (c) a cyclin dependent kinase (CDK) 4/6 inhibitor; wherein the kit does not comprises a KRAS inhibitor. In some embodiments, the kit comprises osimertinib or a salt thereof, cobimetinib or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the cobimetinib or a salt thereof is formulated for oral administration to a subject. In some embodiments, the palbociclib or a salt thereof is formulated for oral administration to a subject.

In some embodiments, osimertinib or a salt thereof, cobimetinib or a salt thereof, and palbociclib or a salt thereof are formulated as one composition. In some embodiments, osimertinib or a salt thereof, cobimetinib or a salt thereof, and palbociclib or a salt thereof are formulated as individual compositions. In some embodiments, osimertinib or a salt thereof and cobimetinib or a salt thereof are formulated as one composition. In some embodiments, osimertinib or a salt thereof and palbociclib or a salt thereof are formulated as one composition. In some embodiments, cobimetinib or a salt thereof and palbociclib or a salt thereof are formulated as one composition. In some embodiments, osimertinib or a salt thereof, cobimetinib or a salt thereof, and palbociclib or a salt thereof are formulated in liquid forms. In some embodiments, osimertinib or a salt thereof, cobimetinib or a salt thereof, and palbociclib or a salt thereof are formulated in solid forms.

In some embodiments, osimertinib or a salt thereof, cobimetinib or a salt thereof, and palbociclib or a salt thereof are administered as one composition. In some embodiments, osimertinib or a salt thereof, cobimetinib or a salt thereof, and palbociclib or a salt thereof are administered separately. In some embodiments, osimertinib or a salt thereof, cobimetinib or a salt thereof, and palbociclib or a salt thereof are administered simultaneously. In some embodiments, osimertinib or a salt thereof, cobimetinib or a salt thereof, and palbociclib or a salt thereof are administered continuously. In some embodiments, osimertinib or a salt thereof, cobimetinib or a salt thereof, and palbociclib or a salt thereof are administered intermittently.

In some embodiments, the kit comprises cetuximab, cobimetinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, cobimetinib or a salt thereof is formulated for oral administration to a subject. In some embodiments, palbociclib or a salt thereof is formulated for oral administration to a subject. In some embodiments, cetuximab, cobimetinib or a salt thereof, and palbociclib or a salt thereof are formulated in different compositions. In some embodiments, cobimetinib or a salt thereof, and palbociclib or a salt thereof are formulated in one composition.

In some embodiments, cetuximab, cobimetinib or a salt thereof, and palbociclib or a salt thereof are formulated as one composition. In some embodiments, cetuximab, cobimetinib or a salt thereof, and palbociclib or a salt thereof are formulated as individual compositions. In some embodiments, cetuximab and palbociclib or a salt thereof are formulated as one composition. In some embodiments, cobimetinib or a salt thereof and palbociclib or a salt thereof are formulated as one composition. In some embodiments, cetuximab, cobimetinib or a salt thereof, and palbociclib or a salt thereof are formulated in liquid forms. In some embodiments, cetuximab, cobimetinib or a salt thereof, and palbociclib or a salt thereof are formulated in solid forms.

In some embodiments, cetuximab, cobimetinib or a salt thereof, and palbociclib or a salt thereof are administered as one composition. In some embodiments, cetuximab, cobimetinib or a salt thereof, and palbociclib or a salt thereof are administered separately. In some embodiments, cetuximab, cobimetinib or a salt thereof, and palbociclib or a salt thereof are administered simultaneously. In some embodiments, cetuximab, cobimetinib or a salt thereof, and palbociclib or a salt thereof are administered continuously. In some embodiments, cetuximab, cobimetinib or a salt thereof, and palbociclib or a salt thereof are administered intermittently. In some embodiments, cetuximab, cobimetinib or a salt thereof, and palbociclib or a salt thereof are administered with different dosing frequencies.

In some embodiments, the kit comprises cetuximab, TAK-733 or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, TAK-733 or a salt thereof is formulated for oral administration to a subject. In some embodiments, palbociclib or a salt thereof is formulated for oral administration to a subject. In some embodiments, cetuximab, TAK-733 or a salt thereof, and palbociclib or a salt thereof are formulated in different compositions. In some embodiments, TAK-733 or a salt thereof, and palbociclib or a salt thereof are formulated in one composition.

In some embodiments, cetuximab, TAK-733 or a salt thereof, and palbociclib or a salt thereof are formulated as one composition. In some embodiments, cetuximab, TAK-733 or a salt thereof, and palbociclib or a salt thereof are formulated as individual compositions. In some embodiments, cetuximab and palbociclib or a salt thereof are formulated as one composition. In some embodiments, TAK-733 or a salt thereof and palbociclib or a salt thereof are formulated as one composition. In some embodiments, cetuximab, TAK-733 or a salt thereof, and palbociclib or a salt thereof are formulated in liquid forms. In some embodiments, cetuximab, TAK-733 or a salt thereof, and palbociclib or a salt thereof are formulated in solid forms.

In some embodiments, cetuximab, TAK-733 or a salt thereof, and palbociclib or a salt thereof are administered as one composition. In some embodiments, cetuximab, TAK-733 or a salt thereof, and palbociclib or a salt thereof are administered separately. In some embodiments, cetuximab, TAK-733 or a salt thereof, and palbociclib or a salt thereof are administered simultaneously. In some embodiments, cetuximab, TAK-733 or a salt thereof, and palbociclib or a salt thereof are administered continuously. In some embodiments, cetuximab, TAK-733 or a salt thereof, and palbociclib or a salt thereof are administered intermittently. In some embodiments, cetuximab, TAK-733 or a salt thereof, and palbociclib or a salt thereof are administered with different dosing frequencies.

In some embodiments, the kit comprises osimertinib or a salt thereof, TAK-733 or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the TAK-733 or a salt thereof is formulated for oral administration to a subject. In some embodiments, the palbociclib or a salt thereof is formulated for oral administration to a subject.

In some embodiments, osimertinib or a salt thereof, TAK-733 or a salt thereof, and palbociclib or a salt thereof are formulated as one composition. In some embodiments, osimertinib or a salt thereof, TAK-733 or a salt thereof, and palbociclib or a salt thereof are formulated as individual compositions. In some embodiments, osimertinib or a salt thereof and TAK-733 or a salt thereof are formulated as one composition. In some embodiments, osimertinib or a salt thereof and palbociclib or a salt thereof are formulated as one composition. In some embodiments, TAK-733 or a salt thereof and palbociclib or a salt thereof are formulated as one composition. In some embodiments, osimertinib or a salt thereof, TAK-733 or a salt thereof, and palbociclib or a salt thereof are formulated in liquid forms. In some embodiments, osimertinib or a salt thereof, TAK-733 or a salt thereof, and palbociclib or a salt thereof are formulated in solid forms.

In some embodiments, osimertinib or a salt thereof, TAK-733 or a salt thereof, and palbociclib or a salt thereof are administered as one composition. In some embodiments, osimertinib or a salt thereof, TAK-733 or a salt thereof, and palbociclib or a salt thereof are administered separately. In some embodiments, osimertinib or a salt thereof, TAK-733 or a salt thereof, and palbociclib or a salt thereof are administered simultaneously. In some embodiments, osimertinib or a salt thereof, TAK-733 or a salt thereof, and palbociclib or a salt thereof are administered continuously. In some embodiments, osimertinib or a salt thereof, TAK-733 or a salt thereof, and palbociclib or a salt thereof are administered intermittently.

In some embodiments, the kit comprises avitinib or a salt thereof, cobimetinib or a salt thereof, and palbociclib or a salt thereof. In some embodiments, the kit does not comprise a KRAS inhibitor. In some embodiments, the kit comprises a pharmaceutical composition comprising avitinib and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, avitinib or a salt thereof, cobimetinib or a salt thereof is formulated for oral administration to a subject. In some embodiments, palbociclib or a salt thereof is formulated for oral administration to a subject. In some embodiments, avitinib or a salt thereof, cobimetinib or a salt thereof, and palbociclib or a salt thereof are formulated in different compositions. In some embodiments, avitinib or a salt thereof, cobimetinib or a salt thereof, and palbociclib or a salt thereof are formulated in one composition.

In some embodiments, avitinib or a salt thereof, cobimetinib or a salt thereof, and palbociclib or a salt thereof are formulated as one composition. In some embodiments, avitinib or a salt thereof, cobimetinib or a salt thereof, and palbociclib or a salt thereof are formulated as individual compositions. In some embodiments, avitinib and palbociclib or a salt thereof are formulated as one composition. In some embodiments, cobimetinib or a salt thereof and palbociclib or a salt thereof are formulated as one composition. In some embodiments, avitinib or a salt thereof, cobimetinib or a salt thereof, and palbociclib or a salt thereof are formulated in liquid forms. In some embodiments, avitinib or a salt thereof, cobimetinib or a salt thereof, and palbociclib or a salt thereof are formulated in solid forms.

In some embodiments, avitinib or a salt thereof, cobimetinib or a salt thereof, and palbociclib or a salt thereof are administered as one composition. In some embodiments, avitinib or a salt thereof, cobimetinib or a salt thereof, and palbociclib or a salt thereof are administered separately. In some embodiments, avitinib or a salt thereof, cobimetinib or a salt thereof, and palbociclib or a salt thereof are administered simultaneously. In some embodiments, avitinib or a salt thereof, cobimetinib or a salt thereof, and palbociclib or a salt thereof are administered continuously. In some embodiments, avitinib or a salt thereof, cobimetinib or a salt thereof, and palbociclib or a salt thereof are administered intermittently. In some embodiments, avitinib or a salt thereof, cobimetinib or a salt thereof, and palbociclib or a salt thereof are administered with different dosing frequencies.

In some embodiments, the kit comprises osimertinib or a salt thereof, binimetinib or a salt thereof, and palbociclib or a salt thereof. In some embodiments, the kit does not comprise a KRAS inhibitor. In some embodiments, the kit comprises a pharmaceutical composition comprising osimertinib and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, osimertinib is formulated for oral administration to a subject. In some embodiments, binimetinib or a salt thereof is formulated for oral administration to a subject. In some embodiments, palbociclib or a salt thereof is formulated for oral administration to a subject. In some embodiments, osimertinib or a salt thereof, binimetinib or a salt thereof, and palbociclib or a salt thereof are formulated in different compositions. In some embodiments, osimertinib or a salt thereof, binimetinib or a salt thereof, and palbociclib or a salt thereof are formulated in one composition.

In some embodiments, osimertinib or a salt thereof, binimetinib or a salt thereof, and palbociclib or a salt thereof are formulated as one composition. In some embodiments, osimertinib or a salt thereof, binimetinib or a salt thereof, and palbociclib or a salt thereof are formulated as individual compositions. In some embodiments, osimertinib and palbociclib or a salt thereof are formulated as one composition. In some embodiments, binimetinib or a salt thereof and palbociclib or a salt thereof are formulated as one composition. In some embodiments, osimertinib or a salt thereof, binimetinib or a salt thereof, and palbociclib or a salt thereof are formulated in liquid forms. In some embodiments, osimertinib or a salt thereof, binimetinib or a salt thereof, and palbociclib or a salt thereof are formulated in solid forms.

In some embodiments, osimertinib or a salt thereof, binimetinib or a salt thereof, and palbociclib or a salt thereof are administered as one composition. In some embodiments, osimertinib or a salt thereof, binimetinib or a salt thereof, and palbociclib or a salt thereof are administered separately. In some embodiments, osimertinib or a salt thereof, binimetinib or a salt thereof, and palbociclib or a salt thereof are administered simultaneously. In some embodiments, osimertinib or a salt thereof, binimetinib or a salt thereof, and palbociclib or a salt thereof are administered continuously. In some embodiments, osimertinib or a salt thereof, binimetinib or a salt thereof, and palbociclib or a salt thereof are administered intermittently. In some embodiments, osimertinib or a salt thereof, binimetinib or a salt thereof, and palbociclib or a salt thereof are administered with different dosing frequencies.

In some embodiments, the kit comprises cetuximab, binimetinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, binimetinib or a salt thereof is formulated for oral administration to a subject. In some embodiments, palbociclib or a salt thereof is formulated for oral administration to a subject. In some embodiments, cetuximab, binimetinib or a salt thereof, and palbociclib or a salt thereof are formulated in different compositions. In some embodiments, binimetinib or a salt thereof, and palbociclib or a salt thereof are formulated in one composition.

In some embodiments, cetuximab, binimetinib or a salt thereof, and palbociclib or a salt thereof are formulated as one composition. In some embodiments, cetuximab, binimetinib or a salt thereof, and palbociclib or a salt thereof are formulated as individual compositions. In some embodiments, cetuximab and palbociclib or a salt thereof are formulated as one composition. In some embodiments, binimetinib or a salt thereof and palbociclib or a salt thereof are formulated as one composition. In some embodiments, cetuximab, binimetinib or a salt thereof, and palbociclib or a salt thereof are formulated in liquid forms. In some embodiments, cetuximab, binimetinib or a salt thereof, and palbociclib or a salt thereof are formulated in solid forms.

In some embodiments, cetuximab, binimetinib or a salt thereof, and palbociclib or a salt thereof are administered as one composition. In some embodiments, cetuximab, binimetinib or a salt thereof, and palbociclib or a salt thereof are administered separately. In some embodiments, cetuximab, binimetinib or a salt thereof, and palbociclib or a salt thereof are administered simultaneously. In some embodiments, cetuximab, binimetinib or a salt thereof, and palbociclib or a salt thereof are administered continuously. In some embodiments, cetuximab, binimetinib or a salt thereof, and palbociclib or a salt thereof are administered intermittently. In some embodiments, cetuximab, binimetinib or a salt thereof, and palbociclib or a salt thereof are administered with different dosing frequencies.

In some embodiments, the kit comprises cetuximab, cobimetinib or a salt thereof, and abemaciclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising abemaciclib or a salt thereof and a pharmaceutically acceptable carrier, excipient, binder, or diluent. In some embodiments, cetuximab is formulated for administration to a subject via intravenous infusion. In some embodiments, cobimetinib or a salt thereof is formulated for oral administration to a subject. In some embodiments, abemaciclib or a salt thereof is formulated for oral administration to a subject. In some embodiments, cetuximab, cobimetinib or a salt thereof, and abemaciclib or a salt thereof are formulated in different compositions. In some embodiments, cobimetinib or a salt thereof, and abemaciclib or a salt thereof are formulated in one composition.

In some embodiments, cetuximab, cobimetinib or a salt thereof, and abemaciclib or a salt thereof are formulated as one composition. In some embodiments, cetuximab, cobimetinib or a salt thereof, and abemaciclib or a salt thereof are formulated as individual compositions. In some embodiments, cetuximab and abemaciclib or a salt thereof are formulated as one composition. In some embodiments, cobimetinib or a salt thereof and abemaciclib or a salt thereof are formulated as one composition. In some embodiments, cetuximab, cobimetinib or a salt thereof, and abemaciclib or a salt thereof are formulated in liquid forms. In some embodiments, cetuximab, cobimetinib or a salt thereof, and abemaciclib or a salt thereof are formulated in solid forms.

In some embodiments, cetuximab, cobimetinib or a salt thereof, and abemaciclib or a salt thereof are administered as one composition. In some embodiments, cetuximab, cobimetinib or a salt thereof, and abemaciclib or a salt thereof are administered separately. In some embodiments, cetuximab, cobimetinib or a salt thereof, and abemaciclib or a salt thereof are administered simultaneously. In some embodiments, cetuximab, cobimetinib or a salt thereof, and abemaciclib or a salt thereof are administered continuously. In some embodiments, cetuximab, cobimetinib or a salt thereof, and abemaciclib or a salt thereof are administered intermittently. In some embodiments, cetuximab, cobimetinib or a salt thereof, and abemaciclib or a salt thereof are administered with different dosing frequencies.

In some embodiments, the kit comprises osimertinib or a salt thereof, trametinib or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the trametinib or a salt thereof is formulated for oral administration to a subject. In some embodiments, the palbociclib or a salt thereof is formulated for oral administration to a subject.

In some embodiments, osimertinib or a salt thereof, trametinib or a salt thereof, and palbociclib or a salt thereof are formulated as one composition. In some embodiments, osimertinib or a salt thereof, trametinib or a salt thereof, and palbociclib or a salt thereof are formulated as individual compositions. In some embodiments, osimertinib or a salt thereof and trametinib or a salt thereof are formulated as one composition. In some embodiments, osimertinib or a salt thereof and palbociclib or a salt thereof are formulated as one composition. In some embodiments, trametinib or a salt thereof and palbociclib or a salt thereof are formulated as one composition. In some embodiments, osimertinib or a salt thereof, trametinib or a salt thereof, and palbociclib or a salt thereof are formulated in liquid forms. In some embodiments, osimertinib or a salt thereof, trametinib or a salt thereof, and palbociclib or a salt thereof are formulated in solid forms.

In some embodiments, osimertinib or a salt thereof, trametinib or a salt thereof, and palbociclib or a salt thereof are administered as one composition. In some embodiments, osimertinib or a salt thereof, trametinib or a salt thereof, and palbociclib or a salt thereof are administered separately. In some embodiments, osimertinib or a salt thereof, trametinib or a salt thereof, and palbociclib or a salt thereof are administered simultaneously. In some embodiments, osimertinib or a salt thereof, trametinib or a salt thereof, and palbociclib or a salt thereof are administered continuously. In some embodiments, osimertinib or a salt thereof, trametinib or a salt thereof, and palbociclib or a salt thereof are administered intermittently.

In some embodiments, the kit comprises cetuximab, trametinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, trametinib or a salt thereof is formulated for oral administration to a subject. In some embodiments, palbociclib or a salt thereof is formulated for oral administration to a subject. In some embodiments, cetuximab, trametinib or a salt thereof, and palbociclib or a salt thereof are formulated in different compositions. In some embodiments, trametinib or a salt thereof, and palbociclib or a salt thereof are formulated in one composition.

In some embodiments, cetuximab, trametinib or a salt thereof, and palbociclib or a salt thereof are formulated as one composition. In some embodiments, cetuximab, trametinib or a salt thereof, and palbociclib or a salt thereof are formulated as individual compositions. In some embodiments, cetuximab and palbociclib or a salt thereof are formulated as one composition. In some embodiments, trametinib or a salt thereof and palbociclib or a salt thereof are formulated as one composition. In some embodiments, cetuximab, trametinib or a salt thereof, and palbociclib or a salt thereof are formulated in liquid forms. In some embodiments, cetuximab, trametinib or a salt thereof, and palbociclib or a salt thereof are formulated in solid forms.

In some embodiments, cetuximab, trametinib or a salt thereof, and palbociclib or a salt thereof are administered as one composition. In some embodiments, cetuximab, trametinib or a salt thereof, and palbociclib or a salt thereof are administered separately. In some embodiments, cetuximab, trametinib or a salt thereof, and palbociclib or a salt thereof are administered simultaneously. In some embodiments, cetuximab, trametinib or a salt thereof, and palbociclib or a salt thereof are administered continuously. In some embodiments, cetuximab, trametinib or a salt thereof, and palbociclib or a salt thereof are administered intermittently. In some embodiments, cetuximab, trametinib or a salt thereof, and palbociclib or a salt thereof are administered with different dosing frequencies.

In some embodiments, the kit comprises a package insert containing instructions regarding indications, usage, dosage, administration, contraindications, other medicaments to be combined with the packaged product, and/or warnings concerning the use of such medicaments.

### BRIEF DESCRIPTION OF THE DRAWINGS

FIG. 1 depicts tumor volume reduction in mice upon treatment with combination therapy in a KRAS^{G12C} NSCLC PDX Model with patient LU-01-0030 FP10. The graph shows the mean tumor volume change over time during the combination treatment: (circle)—control with no treatment; (square)—osimertinib 10 mg/kg, cobimetinib 5 mg/kg, and palbociclib 20 mg/kg daily (p.o.) for 21 days followed by 7 days of holiday; (triangle)—cetuximab 1 mg/kg per week (i.p.), cobimetinib 5 mg/kg and palbociclib 20 mg/kg daily (p.o.) for 21 days followed by 7 days of holiday; and (upside down triangle)—avitinib 100 mg/kg, cobimetinib 5 mg/kg, and palbociclib 20 mg/kg daily (p.o.) for 21 days followed by 7 days of holiday. The dosage unit, mg/kg, refers to dose of the compound per kg of the mouse body weight.

FIG. 2 depicts body weights of the mice upon treatment with combination therapy in a KRAS^{G12C} NSCLC PDX Model with patient LU-01-0030 FP10: (circle)—control with no treatment; (square)—osimertinib 10 mg/kg, cobimetinib 5 mg/kg, and palbociclib 20 mg/kg daily (p.o.) for 21 days followed by 7 days of holiday; (triangle)—cetuximab 1 mg/kg per week (i.p.), cobimetinib 5 mg/kg and palbociclib 20 mg/kg daily (p.o.) for 21 days followed by 7 days of holiday; and (upside down triangle)—avitinib 100 mg/kg, cobimetinib 5 mg/kg, and palbociclib 20 mg/kg daily (p.o.) for 21 days followed by 7 days of holiday. The dosage unit, mg/kg, refers to dose of the compound per kg of the mouse body weight.

FIG. 3 depicts tumor volume reduction in mice upon treatment with combination therapy in a KRAS^{G12V} NSCLC PDX Model with patient LU6419 FP10. The graph shows the mean tumor volume change over time during the combination treatment: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 4 depicts body weights of the mice upon treatment with combination therapy in a KRAS^{G12V} NSCLC PDX Model with patient LU6419 FP10: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 5 depicts tumor volume reduction in mice upon treatment with combination therapy in a KRAS^{G12V} NSCLC PDX Model with patient LU2071. The graph shows the mean tumor volume change over time during the combination treatment: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 6 depicts body weights of the mice upon treatment with combination therapy in a KRAS^{G12V} NSCLC PDX Model with patient LU2071. The graph shows the mean tumor volume change over time during the combination treatment: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 7 depicts tumor volume reduction in mice upon treatment with combination therapy in a KRAS^{G12C} NSCLC PDX Model with patient LU6405. The graph shows the mean tumor volume change over time during the combination treatment: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 8 depicts body weights of the mice upon treatment with combination therapy in a KRAS^{G12C} NSCLC PDX Model with patient LU6405. The graph shows the mean tumor volume change over time during the combination treatment: (square)—control with no treatment; (circle)—osimertinib 10 mg/kg, cobimetinib 5 mg/kg, and palbociclib 20 mg/kg daily (p.o.) for 21 days followed by 7 days of holiday; and (diamond)—cetuximab 1 mg/kg per week (i.p.), cobimetinib 5 mg/kg and palbociclib 20 mg/kg daily (p.o.) for 21 days followed by 7 days of holiday. The dosage unit, mg/kg, refers to dose of the compound per kg of the mouse body weight.

FIG. 9. depicts tumor volume reduction upon treatment with combination therapy in a KRAS^{G12C} NSCLC CDX model H358. The graph shows the mean tumor volume change over time during the combination treatment: (solid circle)—control with no treatment for 28 days; (solid triangle)—cisplatin 4 mg/kg per week (i.p.) for 21 days followed by 7 days of holiday; (open circle)—TAK-733 10 mg/kg and palbociclib 20 mg/kg daily (p.o.) for 21 days followed by 7 days of holiday; (open triangle)—cobimetinib 5 mg/kg and palbociclib 20 mg/kg daily (p.o.) for 21 days followed by 7 days of holiday; (solid diamond)—osimertinib 10 mg/kg, cobimetinib 5 mg/kg, and palbociclib 20 mg/kg daily (p.o.) for 21 days followed by 7 days of holiday; (solid square)—cetuximab 1 mg/kg per week (i.p.), TAK-733 30 mg/kg and palbociclib 20 mg/kg daily (p.o.) for 21 days followed by 7 days of holiday; after Day 10, TAK-733 reduced to 3 mg/kg; (open diamond)—cetuximab 1 mg/kg per week (i.p.), cobimetinib 5 mg/kg and palbociclib 20 mg/kg daily (p.o.) for 21 days followed by 7 days of holiday; and (open square)—cetuximab 1 mg/kg per week (i.p.), TAK-733 30 mg/kg and palbociclib 20 mg/kg daily (p.o.) for 21 days followed by 7 days of holiday.

FIG. 10 depicts body weights of the mice upon treatment with combination therapy in a KRAS^{G12C} NSCLC CDX model H358: (solid circle)—control with no treatment for 28 days; (solid triangle)—cisplatin 4 mg/kg per week (i.p.) for 21 days followed by 7 days of holiday; (open circle)—TAK-733 10 mg/kg and palbociclib 20 mg/kg daily (p.o.) for 21 days followed by 7 days of holiday; (open triangle)—cobimetinib 5 mg/kg and palbociclib 20 mg/kg daily (p.o.) for 21 days followed by 7 days of holiday; (solid diamond)—osimertinib 10 mg/kg, cobimetinib 5 mg/kg, and palbociclib 20 mg/kg daily (p.o.) for 21 days followed by 7 days of holiday; (solid square)—cetuximab 1 mg/kg per week (i.p.), TAK-733 30 mg/kg and palbociclib 20 mg/kg daily (p.o.) for 21 days followed by 7 days of holiday; after Day 10, TAK-733 reduced to 3 mg/kg; (open diamond)—cetuximab 1 mg/kg per week (i.p.), cobimetinib 5 mg/kg and palbociclib 20 mg/kg daily (p.o.) for 21 days followed by 7 days of holiday; and (open square)—cetuximab 1 mg/kg per week (i.p.), TAK-733 30 mg/kg and palbociclib 20 mg/kg daily (p.o.) for 21 days followed by 7 days of holiday.

### DETAILED DESCRIPTION

The present description is based on the inventor's data showing that a combination of an epidermal growth factor receptor inhibitor (such as osimertinib, avitinib or cetuximab), a mitogen-activated protein kinase 1/2 inhibitor (such as cobimetinib, trametinib, binimetinib, or TAK-733) and a cyclin dependent kinase 4/6 inhibitor (such as palbociclib or abemaciclib) provides a robust therapy for a method of treating or delaying progression of cancer with a KRAS mutation. Particularly, such a combination therapy does not require a KRAS inhibitor. The combination therapy described herein has surprisingly demonstrated a synergistic effect toward cancers with a KRAS mutation and a robust efficacy in inhibiting the tumor growth by up to 100% and in reducing the tumor by over 90% in well-established animal models, despite the fact that none of the compounds in the combination is an inhibitor of the mutant KRAS. The description also provides compositions and kits that can be used for carrying out this combination therapy.

### I. DEFINITIONS

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a subject “at risk” of developing a disease may or may not have detectable disease, or symptoms of disease, and may or may not have displayed detectable disease or symptoms of disease prior to the treatment methods described herein. A subject “at risk” has one or more risk factors, which are measurable parameters that correlate with development of a disease (such as cancer), as described herein and known in the art. A subject “at risk” may have one or more risk factors. A subject having one or more risk factors has higher probability of developing the disease than a subject without one or more risk factors.

As used herein, “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at a second site derived from any of the above types of cancer. Included in this definition are benign and malignant cancers as well as dormant tumors or micrometastases.

The terms “neoplastic cell”, “tumor cell”, or “cancer cell”, used either in the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, “delaying progression” of a disease means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

As used herein, the term “effective amount” or “therapeutically effective amount” of a substance is at least the minimum concentration required to effect a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “inhibitor” or “antagonist” refers to biological or chemical substance that interferes with or otherwise reduces the physiological and/or biochemical action of another biological or chemical molecule. In some embodiments, the inhibitor or antagonist specifically binds to the other molecule.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “pharmaceutically acceptable salt” is a salt form that is non-toxic, biologically tolerable, or otherwise biologically suitable for administration to the subject. See generally Berge et al. (1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

As used herein, a “tissue sample” or “cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ, such as a cancer or tumor tissue. The tissue sample may contain compounds which are not naturally intermixed with the tissue in nature such as preservatives, anticoagulants, buffers, fixatives, nutrients, antibiotics, or the like.

As used herein, the term “treatment” refers to clinical intervention designed to have beneficial and desired effects to the natural course of the individual or cell being treated during the course of clinical pathology. For the purpose of this disclosure, desirable effects of treatment include, without limitation, decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, increasing cancer cell-killing, decreasing symptoms resulting from the disease, preventing spread of diseases, preventing recurrence of disease, increasing the quality of life of those suffering from the disease, decreasing the dose of other medications required to treat the disease, delaying the progression of the disease, and/or prolonging survival of individuals.

### II. METHODS OF TREATING AND DELAYING PROGRESSION OF CANCER WITH KRAS MUTATION

Provided herein are methods for treating or delaying progression of cancer in a subject comprising administering to the subject an effective amount of (a) an epidermal growth factor receptor (EGFR) inhibitor (such as osimertinib, avitinib, or cetuximab); (b) a mitogen-activated protein kinase (MEK) 1/2 inhibitor (such as cobimetinib, trametinib, binimetinib, or TAK-733); and (c) a cyclin dependent kinase (CDK) 4/6 inhibitor (such as palbociclib or abemaciclib); wherein the subject has cancer that has a KRAS mutation or is at risk of developing cancer that has a KRAS mutation.

In some embodiments, (a) is a small molecule or antibody (or antigen-binding fragment thereof) which specifically binds to EGFR or a ligand thereof, and is optionally selected from one or more of cetuximab, panitumumab, zalutumumab, nimotuzumab, matuzumab, erlotinib, gefitinib, afatinib, lapatinib, osimertinib, avitinib, brigatinib and icotinib (including salt forms of any of the compounds). In some embodiments, (b) is a small molecule or antibody (or antigen-binding fragment thereof) which specifically binds to a MEK 1/2 or a ligand thereof, and is optionally selected from one or more of trametinib, selumetinib, binimetinib, TAK-733, CI-1040, PD0325901, MEK162, AZD8330, GDC-0623, refametinib, pimasertib, RO4987655, RO5126766, WX-554, HL-085 and cobimetinib (including salt forms of any of the compounds). In some embodiments, (c) is a small molecule or antibody (or antigen-binding fragment thereof) which specifically binds to a CDK 4/6 or a ligand thereof, and is optionally selected from one or more of palbociclib, ribociclib and abemaciclib (including salt forms of any of the compounds). In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the subject had received a KRAS inhibitor during a previous treatment cycle. In some embodiments, the subject has not received a KRAS inhibitor during a previous treatment cycle.

In one aspect, the method disclosed here can be used to treat or delay progression of a cancer that has a KRAS mutation. KRAS is a GTPase and KRAS mutations have been found in various human cancers, including but not limited to, pancreatic carcinomas, colon carcinomas, lung carcinomas, biliary tract malignancies, endometrial cancer, cervical cancer, bladder cancer, liver cancer, myeloid leukemia and breast cancer. Oncogenic forms of the KRAS gene are particularly prevalent in pancreatic cancer, colorectal cancer and lung cancer. KRAS has been reported to be mutated at several sites, but the vast majority of mutations occur at the Gly residue of codon 12 and codon 13. Common mutations include G12C, G12D, G12V, G12A, G12S, G12R and G13D. See Jia et al. (2017) Oncol. Lett. 14, 6525. Detection of these mutations can be performed using conventional methods, such as the non-limiting example reported in Lasota et al. (2015) Am. J. Surg. Pathol. 38, 1235. In some embodiments, KRAS mutation is detected in tissue or cell samples containing cancer cells from a subject. In some embodiments, the KRAS mutation is a somatic mutation. In some embodiments, the method is used to treat or delay progression of a cancer that has a KRAS G12C mutation. In some embodiments, the method is used to treat or delay progression of a cancer that has a KRAS G12V mutation. In some embodiments, the method is used to treat or delay progression of a cancer that has a KRAS G12D mutation. In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the subject had received a KRAS inhibitor during a previous treatment cycle. In some embodiments, the subject has not received a KRAS inhibitor during a previous treatment cycle.

In some embodiments, the cancer is an adenocarcinoma, a squamous cell carcinoma, an adenosquamous carcinoma, an anaplastic carcinoma, a large cell carcinoma, and a small cell carcinoma. In some embodiments, the cancer is melanoma and carcinoma, such as an epithelial neoplasm, a squamous cell neoplasm, a basal cell neoplasm, a transitional cell carcinoma, an adenocarcinoma, an adnexal or skin appendage neoplasm, a nucoepidermoid neoplasm, a cystic, mucinous, or Serous neoplasm, a ductal, lobular, or medullary neoplasm, an acinar cell neoplasm, and a complex epithelial neoplasm. In some embodiments, the carcinoma is a colon cancer, a gastric cancer, a lung cancer, a breast cancer, a pancreatic cancer, an oral cancer, a prostate cancer, a germline cancer, a rectal cancer, a liver cancer, a kidney cancer, and an ovarian cancer. In some embodiments, the cancer is a pancreatic cancer, colorectal cancer or lung cancer. In some embodiments, the cancer is NSCLC.

Also provided here are methods for treating or delaying progression of cancer in a subject comprising administering to the subject an effective amount of osimertinib or a salt thereof, cobimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of cetuximab, cobimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of cetuximab, TAK-733 or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of osimertinib or a salt thereof, TAK-733 or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of avitinib or a salt thereof, cobimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of osimertinib or a salt thereof, binimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of cetuximab, binimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of cetuximab, cobimetinib or a salt thereof, and abemaciclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of cetuximab, binimetinib or a salt thereof, and palbociclib or a salt thereof, wherein the subject has cancer that has a KRAS mutation or is at risk of developing cancer that has a KRAS mutation. In another aspect, provided here are methods for treating or delaying progression of cancer in a subject comprising administering to the subject an effective amount of osimertinib, binimetinib or a salt thereof, and palbociclib or a salt thereof, wherein the subject has cancer that has a KRAS mutation or is at risk of developing cancer that has a KRAS mutation.

Osimertinib is an oral, third-generation EGFR inhibitor approved for treating non-small cell lung cancer harboring EGFR mutations by U.S. FDA and European Commission (EC). Osimertinib targets EGFR tyrosine kinase inhibitor (TKI)-sensitizing mutations and particularly T790M that often contributes to acquired resistance to EGFR TKI therapy. Cobimetinib is a MEK inhibitor approved by U.S. FDA to be used in combination with vemurafenib, a BRAF inhibitor, for treating metastatic melanoma with BRAF V600E or V600K mutation. Cobimetinib and vemurafenib target different components of the MAPK/ERK pathway: MEK and BRAF respectively. Palbociclib was a CDK4/6 inhibitor approved by U.S. FDA for treating hormone receptor (HR) positive, human epidermal growth factor receptor 2 (HER2) negative advanced or metastatic breast cancer in combination with an aromatase inhibitor as initial endocrine based therapy in postmenopausal women. Cetuximab is a chimeric monoclonal antibody given by intravenous infusion and an EGFR inhibitor approved by U.S. FDA in 2009 for treatment of colon cancer with wild-type KRAS. TAK-733 is an orally bioavailable, non-ATP-competitive small-molecule MEK1/2 inhibitor that completed a Phase I clinical study. Avitinib is an orally bioavailable, irreversible EGFR inhibitor that has finished the Phase II clinical trials in China, and has shown similar positive results to osimertinib. See, Thoracic Cancer, Volume 11, Issue 10, pp 2775-2781. Binimetinib is an MEK1/2 inhibitor approved by the FDA in 2018, for the treatment of patients with unresectable or metastatic BRAF V600E or V600K mutation-positive melanoma in combination with encorafenib. Abemaciclib is a U.S. FDA approved, orally available cyclin-dependent kinase (CDK) inhibitor that targets the CDK4 (cyclin D1) and CDK6 (cyclin D3) cell cycle pathway, with potential antineoplastic activity. The structures of osimertinib, TAK-733, cobimetinib, avitinib, binimetinib, abemaciclib and palbociclib are shown below.

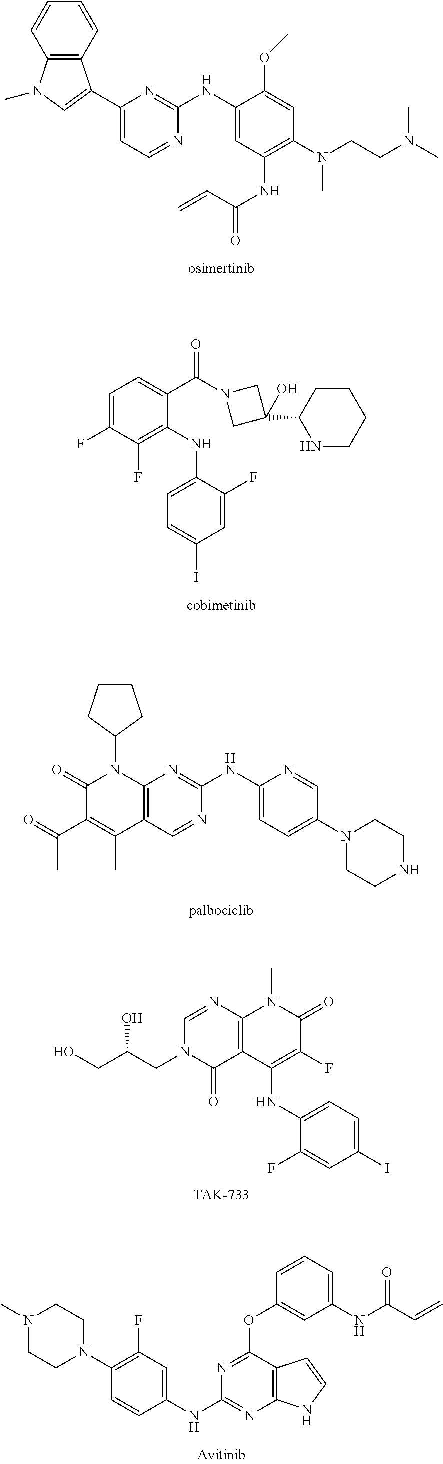

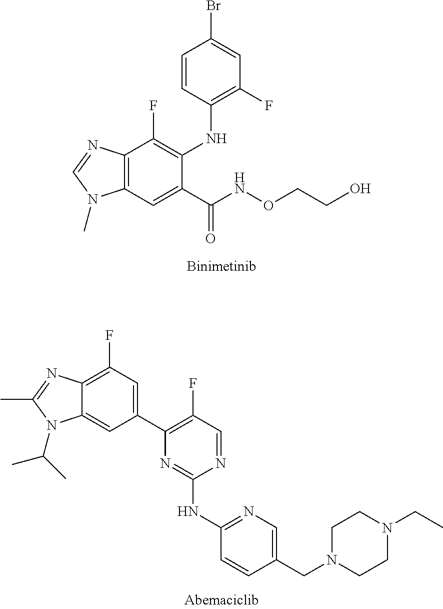 [SMILES: Cn1cnc2c(F)c(Nc3ccc(Br)cc3F)c(C(=O)NOCCO)cc21 ; CCN1CCN(Cc2ccc(Nc3ncc(F)c(-c4cc(F)c5nc(C)n(C(C)C)c5c4)n3)nc2)CC1]

In some embodiments, osimertinib, avitinib, cobimetinib, binimetinib, TAK-733, abemaciclib or palbociclib is administered alone or in combination in a salt form. In some embodiments, the salts are pharmaceutically acceptable salts. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, mesylates propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, the pharmaceutically acceptable salts are fumarates. In some embodiments, the pharmaceutically acceptable salts are mesylates. In some embodiments, palbociclib is administered in a fumarate salt form. In some embodiments, cobimetinib is administered in a fumarate salt form. In some embodiments, palcociclib is administered in a hemifumarate salt form. In some embodiments, cobimetinib is administered in a hemifumarate salt form. In some embodiments, osimertinib or abemaciclib is administered in a mesylate salt form. In some embodiments, avitinib is administered in a maleate salt form. In some embodiments, osimertinib, avitinib, cobimetinib, binimetinib, TAK-733, trametinib, abemaciclib, or palbociclib is administered in a solvate form.

The method may comprise administering any compositions or kits described herein.

In another aspect, the method comprises administering an effective amount of osimertinib or a salt thereof, cobimetinib or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of osimertinib or a salt thereof is in the amount of about 40 mg, about 80 mg, or about 160 mg. In some embodiments, the daily dosage of osimertinib or a salt thereof is in the amount of less than about 40 mg, less than about 80 mg, or less than about 160 mg. In some embodiments, the dosage of osimertinib or a salt thereof is in the amount of about 40-160 mg. In some embodiments, the daily dosage of osimertinib or a salt thereof is in the amount of about 0.6-2.7 mg/kg. In some embodiments, osimertinib is in its mesylate salt form. In some embodiments, osimertinib is in its mesylate salt form. The amounts of the inhibitor described herein and throughout the specification refer to the amount of the inhibitor without taking into consideration of the weight of the counterions if the inhibitor exists in a salt form. For example, the term “80 mg of osimertinib” could include, without limitation, 80 mg of osimertinib in a salt-free form or 95.4 mg of osimertinib mesylate.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, less than about 40 mg, or less than about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the daily dosage of palbociclib or a salt thereof is in the amount of about 0.25-0.5 mg/kg, 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of osimertinib or a solvate or a salt thereof, cobimetinib or a solvate or a salt thereof, and palbociclib or a solvate or a salt thereof.

In some embodiments, the daily dosage of osimertinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of osimertinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In some embodiments, the daily dosage of cobimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of cobimetinib or a salt thereof in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In some embodiments, the daily dosage of palbociclib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% c, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of palbociclib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In another aspect, the method comprises administering an effective amount of avitinib or a salt thereof, cobimetinib or a salt thereof, and palbociclib or a salt thereof. In some embodiments, the daily dosage of avitinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of avitinib or a salt thereof is in the amount of about 40 mg, about 80 mg, or about 160 mg. In some embodiments, the daily dosage of avitinib or a salt thereof is in the amount of less than about 40 mg, less than about 80 mg, or less than about 160 mg. In some embodiments, the dosage of avitinib or a salt thereof is in the amount of about 40-160 mg. In some embodiments, the daily dosage of avitinib or a salt thereof is in the amount of about 0.6-2.7 mg/kg. In some embodiments, avitinib is in its maleate salt form. The amounts of the inhibitor described herein and throughout the specification refer to the amount of the inhibitor without taking into consideration of the weight of the counterions if the inhibitor exists in a salt form. For example, the term “80 mg of avitinib” could include, without limitation, 80 mg of avitinib in a salt-free form or 99 mg of avitinib maleate.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, less than about 40 mg, or less than about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the daily dosage of palbociclib or a salt thereof is in the amount of about 0.25-0.5 mg/kg, 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg.

In some embodiments, the daily dosage of avitinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of avitinib, cobimetinib, and palbociclib or salts of the foregoing. In some embodiments, the daily dosage of avitinib or a salt thereof is in the amount that is about at less than 2 0%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of avitinib, cobimetinib, and palbociclib or salts of the foregoing.

In some embodiments, the daily dosage of cobimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of avitinib, cobimetinib, and palbociclib or salts of the foregoing. In some embodiments, the daily dosage of cobimetinib or a salt thereof in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of avitinib, cobimetinib, and palbociclib or salts of the foregoing.

In some embodiments, the daily dosage of palbociclib or a salt thereof is in the amount that is about at greater than about 200%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of avitinib, cobimetinib, and palbociclib or salts of the foregoing. In some embodiments, the daily dosage of palbociclib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of avitinib, cobimetinib, and palbociclib or salts of the foregoing.

In another aspect, the method comprises administering an effective amount of cetuximab, binimetinib or a salt thereof, and palbociclib or a salt thereof. In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or less than about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In some embodiments, the daily dosage of palbociclib or a salt thereof is in the amount of about 0.25-0.5 mg/kg, 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg.

In another aspect, the method comprises administering an effective amount of osimertinib or a salt thereof, binimetinib or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of osimertinib or a salt thereof is in the amount of about 40 mg, about 80 mg, or about 160 mg. In some embodiments, the daily dosage of osimertinib or a salt thereof is in the amount of less than about 40 mg, less than about 80 mg, or less than about 160 mg. In some embodiments, the dosage of osimertinib or a salt thereof is in the amount of about 40-160 mg. In some embodiments, the daily dosage of osimertinib or a salt thereof is in the amount of about 0.6-2.7 mg/kg. In some embodiments, osimertinib is in its mesylate salt form. In some embodiments, osimertinib is in its mesylate salt form. The amounts of the inhibitor described herein and throughout the specification refer to the amount of the inhibitor without taking into consideration of the weight of the counterions if the inhibitor exists in a salt form. For example, the term “80 mg of osimertinib” could include, without limitation, 80 mg of osimertinib in a salt-free form or 95.4 mg of osimertinib mesylate.

In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or less than about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In some embodiments, the daily dosage of palbociclib or a salt thereof is in the amount of about 0.25-0.5 mg/kg, 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg.

In some embodiments, the daily dosage of osimertinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, binimetinib, and palbociclib or salts of the foregoing. In some embodiments, the daily dosage of osimertinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, binimetinib, and palbociclib or salts of the foregoing.

In some embodiments, the daily dosage of binimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, binimetinib, and palbociclib or salts of the foregoing. In some embodiments, the daily dosage of binimetinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, binimetinib, and palbociclib or salts of the foregoing. In some embodiments, the daily dosage of palbociclib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, binimetinib, and palbociclib or salts of the foregoing. In some embodiments, the daily dosage of avitinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, binimetinib, and palbociclib or salts of the foregoing.

In another aspect, the method comprises administering an effective amount of cetuximab, cobimetinib or a salt thereof, and abemaciclib or a salt thereof. In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, less than about 40 mg, or less than about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the dosage of abemaciclib is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, about 3-5 mg/kg, about 4-5 mg/kg, about 4-6 mg/kg, about 3-6 mg/kg, or about 3-7 mg/kg. In some embodiments, the daily dosage of abemaciclib or a salt thereof is in the amount of about 100 mg, about 200 mg, about 300 mg, or about 400 mg. In some embodiments, the daily dosage of abemaciclib or a salt thereof is in the amount of about 50 mg twice daily, about 100 mg twice daily, about 150 mg twice daily, or about 200 mg twice daily. In some embodiments, the daily dosage of abemaciclib or a salt thereof is in the amount of less than about 300 mg, or less than about 400 mg. In some embodiments, the daily dosage of abemaciclib or a salt thereof is in the amount of about 300-400 mg. In some embodiments, the daily dosage of abemaciclib or a salt thereof is in the amount of about 4-6 mg/kg. In some embodiments, abemaciclib is in its mesylate salt form. The amounts of the inhibitor described herein and throughout the specification refer to the amount of the inhibitor without taking into consideration of the weight of the counterions if the inhibitor exists in a salt form. For example, the term “150 mg of abemaciclib” could include, without limitation, 150 mg of abemaciclib in a salt-free form or 178.7 mg of abemaciclib mesylate.

In another aspect, the method comprises administering an effective amount of cetuximab, cobimetinib or a salt thereof, and palbociclib or a salt thereof. In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, less than about 40 mg, or less than about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of cetuximab, cobimetinib or a solvate or a salt thereof, and palbociclib or a solvate or a salt thereof.

In another aspect, the method comprises administering an effective amount of cetuximab, TAK-733 or a salt thereof and palbociclib or a salt thereof. In some embodiments, the daily dosage of TAK-733 or a salt thereof is in the amount of about 0.001-1 mg/kg, about 0.001-0.002 mg/kg, about 0.002-0.005 mg/kg, about 0.005-0.01 mg/kg, about 0.01-0.05 mg/kg, about 0.05-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of TAK-733. In some embodiments, the daily dosage of TAK-733 or a salt thereof is in the amount of about 10 mg, about 15 mg, about 20 mg, or about 25 mg of TAK-733. In some embodiments, the daily dosage of TAK-733 or a salt thereof is in the amount of less than about 10 mg, less than about 15 mg, less than about 20 mg, or less than about 25 mg of TAK-733. In some embodiments, the daily dosage of TAK-733 or a salt thereof is in the amount of about 0.1-25 mg, about 0.1-1 mg, about 1-5 mg, about 5-10 mg, about 8-16 mg, about 10-15 mg, about 15-20 mg, or about 20-25 mg of TAK-733.

In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, less than about 50 mg, less than about 75 mg, less than about 100 mg, or less than about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of cetuximab, TAK-733 or a solvate or a salt thereof, and palbociclib or a solvate or a salt thereof.

In another aspect, the method comprises administering an effective amount of osimertinib or a salt thereof, TAK-733 or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg of osimertinib. In some embodiments, the daily dosage of osimertinib or a salt thereof is in the amount of about 20 mg, about 40 mg, about 80 mg, or about 160 mg of osimertinib. In some embodiments, the daily dosage of osimertinib or a salt thereof is in the amount of less than about 20 mg, less than about 40 mg, less than about 80 mg, or less than about 160 mg of osimertinib. In some embodiments, the dosage of osimertinib or a salt thereof is in the amount of about 20-240 mg, about 20-40 mg, about 40-80 mg, about 80-160 mg, about 160-240 mg or about 40-160 mg of osimertinib. In some embodiments, the daily dosage of osimertinib or a salt thereof is in the amount of about 0.6-2.7 mg/kg of osimertinib. In some embodiments, osimertinib is in its mesylate salt form.

In some embodiments, the daily dosage of TAK-733 or a salt thereof is in the amount of about 0.001-1 mg/kg, about 0.001-0.002 mg/kg, about 0.002-0.005 mg/kg, about 0.005-0.01 mg/kg, about 0.01-0.05 mg/kg, about 0.05-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of TAK-733. In some embodiments, the daily dosage of TAK-733 or a salt thereof is in the amount of about 10 mg, about 15 mg, about 20 mg, or about 25 mg of TAK-733. In some embodiments, the daily dosage of TAK-733 or a salt thereof is in the amount of less than about 10 mg, less than about 15 mg, less than about 20 mg, or less than about 25 mg of TAK-733. In some embodiments, the daily dosage of TAK-733 or a salt thereof is in the amount of about 0.1-25 mg, about 0.1-1 mg, about 1-5 mg, about 5-10 mg, about 8-16 mg, about 10-15 mg, about 15-20 mg, or about 20-25 mg of TAK-733.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, less than about 50 mg, less than about 50 mg, less than about 75 mg, less than about 100 mg, or less than about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of osimertinib or a solvate or a salt thereof, TAK-733 or a solvate or a salt thereof, and palbociclib or a solvate or a salt thereof.

In another aspect, the method comprises administering an effective amount of cetuximab, trametinib or a salt thereof and palbociclib or a salt thereof. In some embodiments, the daily dosage of trametinib or a salt thereof is in the amount of about 0.01-1 mg/kg, about 0.01-0.02 mg/kg, about 0.02-0.03 mg/kg, about 0.03-0.05 mg/kg, about 0.05-0.08 mg/kg, about 0.08-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of trametinib. In some embodiments, the daily dosage of trametinib or a salt thereof is in the amount of about 0.5 mg, about 1 mg, about 2 mg, or about 4 mg of trametinib. In some embodiments, the daily dosage of trametinib or a salt thereof is in the amount of less than about 0.5 mg, less than about 1 mg, less than about 2 mg, or less than about 4 mg of trametinib. In some embodiments, the daily dosage of trametinib or a salt thereof is in the amount of about 0.1-25 mg, about 0.1-0.5 mg, about 0.5-1 mg, about 1-2 mg, about 2-4 mg, about 4-10 mg or about 0.5-2 mg of trametinib.

In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, less than about 50 mg, less than about 50 mg, less than about 75 mg, less than about 100 mg, or less than about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of cetuximab, TAK-733 or a solvate or a salt thereof, and palbociclib or a solvate or a salt thereof.

In another aspect, the method comprises administering an effective amount of osimertinib or a salt thereof, trametinib or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg of osimertinib. In some embodiments, the daily dosage of osimertinib or a salt thereof is in the amount of about 20 mg, about 40 mg, about 80 mg, or about 160 mg of osimertinib. In some embodiments, the daily dosage of osimertinib or a salt thereof is in the amount of less than about 20 mg, less than about 40 mg, less than about 80 mg, or less than about 160 mg of osimertinib. In some embodiments, the dosage of osimertinib or a salt thereof is in the amount of about 20-240 mg, about 20-40 mg, about 40-80 mg, about 80-160 mg, about 160-240 mg or about 40-160 mg of osimertinib. In some embodiments, the daily dosage of osimertinib or a salt thereof is in the amount of about 0.6-2.7 mg/kg of osimertinib. In some embodiments, osimertinib is in its mesylate salt form.

In some embodiments, the daily dosage of trametinib or a salt thereof is in the amount of about 0.01-1 mg/kg, about 0.01-0.02 mg/kg, about 0.02-0.03 mg/kg, about 0.03-0.05 mg/kg, about 0.05-0.08 mg/kg, about 0.08-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of trametinib. In some embodiments, the daily dosage of trametinib or a salt thereof is in the amount of about 0.5 mg, about 1 mg, about 2 mg, or about 4 mg of trametinib. In some embodiments, the daily dosage of trametinib or a salt thereof is in the amount of less than about 0.5 mg, less than about 1 mg, less than about 2 mg, or less than about 4 mg of trametinib. In some embodiments, the daily dosage of trametinib or a salt thereof is in the amount of about 0.1-25 mg, about 0.1-0.5 mg, about 0.5-1 mg, about 1-2 mg, about 2-4 mg, about 4-10 mg or about 0.5-2 mg of trametinib.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, less than about 50 mg, less than about 50 mg, less than about 75 mg, less than about 100 mg, or less than about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of osimertinib or a solvate or a salt thereof, trametinib or a solvate or a salt thereof, and palbociclib or a solvate or a salt thereof.

In another aspect, the method provides administering an effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib and abemaciclib) until disease progression or unacceptable toxicity. In some embodiments, the method provides administering an effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib and abemaciclib) for at least about 1-2 weeks, about 2-3 weeks, about 3-4 weeks, about 4-5 weeks, about 5-6 weeks, about 6-7 weeks, about 7-8 weeks, about 8-9 weeks, about 9-10 weeks, about 2-3 months, about 3-4 months, about 4-5 months, about 5-6 months, about 6-12 months, or about 12-24 months. In some embodiments, the effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib and abemaciclib) is administered for 21 consecutive days followed by 7 days off to comprise a complete cycle of 28 days. In some embodiments, the subject is administered an effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib and abemaciclib) for one, two, three, four, five, six, seven, eight, nine, ten or more cycles of 28 days. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are administered for different durations.

In another aspect, the effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib or abemaciclib) are formulated as one composition. In another aspect, the effective amount of an EGFR inhibitor (e.g., osimertinib, avitinib, or cetuximab), a MEK 1/2 inhibitor (e.g., cobimetinib, trametinib, binimetinib, or TAK-733) and a CDK 4/6 inhibitor (e.g., palbociclib or abemaciclib) are formulated separately. In some embodiments, osimertinib or a salt thereof, cobimetinib or a salt thereof, and palbociclib or a salt thereof are formulated as one composition or separate compositions for oral administration. In some embodiments, osimertinib or a salt thereof, TAK-733 or a salt thereof, and palbociclib or a salt thereof are formulated as one composition or separate compositions for oral administration. In some embodiments, osimertinib or a salt thereof, trametinib or a salt thereof, and palbociclib or a salt thereof are formulated as one composition or separate compositions for oral administration. In some embodiments, cobimetinib or a salt thereof and palbociclib or a salt thereof are formulated as one composition or separate compositions for oral administration. In some embodiments, TAK-733 or a salt thereof and palbociclib or a salt thereof are formulated as one composition or separate compositions for oral administration. In some embodiments, trametinib or a salt thereof and palbociclib or a salt thereof are formulated as one composition or separate compositions for oral administration. For oral administration, the method may comprise formulating the compounds in a solid form, such as a tablet or capsule, or as a solution, emulsion, or suspension. To prepare the oral compositions, the compounds may be formulated to yield a daily dosage described herein. In some embodiments, cetuximab is formulated for intravenous infusion.

In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated in the same form. In some embodiments, osimertinib or a salt thereof, cobimetinib or a salt thereof, and/or palbociclib or a salt thereof are formulated in a solid form, such as a tablet or capsule. In some embodiments, osimertinib or a salt thereof, TAK-733 or a salt thereof, and/or palbociclib or a salt thereof are formulated in a solid form, such as a tablet or capsule. In some embodiments, osimertinib or a salt thereof, trametinib or a salt thereof, and/or palbociclib or a salt thereof are formulated in a solid form, such as a tablet or capsule. In some embodiments, cetuximab, cobimetinib or a salt thereof, and/or palbociclib or a salt thereof are formulated in a liquid form, such as suspensions, solutions, emulsions, or syrups, or may be lyophilized. In some embodiments, cetuximab, TAK-733 or a salt thereof, and/or palbociclib or a salt thereof are formulated in a liquid form, such as suspensions, solutions, emulsions, or syrups, or may be lyophilized. In some embodiments, cetuximab, trametinib or a salt thereof, and/or palbociclib or a salt thereof are formulated in a liquid form, such as suspensions, solutions, emulsions, or syrups, or may be lyophilized. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated in the different forms.

In another aspect, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are administered simultaneously or intermittently. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated as one composition and administered as one composition. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered simultaneously. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered intermittently. In some embodiments, the EGFR inhibitor (e.g., osimertinib, avitinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered with different dosing frequencies.

In another aspect, the method comprises administering an effective amount of osimertinib or a salt thereof, cobimetinib or a salt thereof, and palbociclib or a salt thereof. In some embodiments, osimertinib or a salt thereof is administered before cobimetinib or a salt thereof and palbociclib or a salt thereof. In some embodiments, cobimetinib or a salt thereof is administered before osimertinib or a salt thereof and palbociclib or a salt thereof. In some embodiments, palbociclib or a salt thereof is administered before osimertinib or a salt thereof and cobimetinib or a salt thereof. In some embodiments, osimertinib or a salt thereof and cobimetinib or a salt thereof are administered together in the same or separate compositions. In some embodiments, osimertinib or a salt thereof and palbociclib or a salt thereof are administered together in the same or separate compositions. In some embodiments, cobimetinib or a salt thereof and palbociclib or a salt thereof are administered together in the same or separate compositions.

In another aspect, the method comprises administering an effective amount of cetuximab, cobimetinib or a salt thereof, and palbociclib or a salt thereof. In some embodiments, cetuximab is administered before cobimetinib or a salt thereof and palbociclib or a salt thereof. In some embodiments, cobimetinib or a salt thereof is administered before cetuximab and palbociclib or a salt thereof. In some embodiments, palbociclib or a salt thereof is administered before cetuximab and cobimetinib or a salt thereof. In some embodiments, cetuximab and cobimetinib or a salt thereof are administered together in the same or separate compositions. In some embodiments, cetuximab and palbociclib or a salt thereof are administered together in the same or separate compositions. In some embodiments, cobimetinib or a salt thereof and palbociclib or a salt thereof are administered together in the same or separate compositions. In some embodiments, cetuximab is administered in a separate composition from cobimetinib or a salt thereof, and palbociclib or a salt thereof. In some embodiments, cetuximab is administered every week or every two weeks, while cobimetinib or a salt thereof and palbociclib or a salt thereof are administered on a daily basis.

In another aspect, the method comprises administering an effective amount of osimertinib or a salt thereof, TAK-733 or a salt thereof, and palbociclib or a salt thereof. In some embodiments, osimertinib or a salt thereof is administered before TAK-733 or a salt thereof and palbociclib or a salt thereof. In some embodiments, TAK-733 or a salt thereof is administered before osimertinib or a salt thereof and palbociclib or a salt thereof. In some embodiments, palbociclib or a salt thereof is administered before osimertinib or a salt thereof and TAK-733 or a salt thereof. In some embodiments, osimertinib or a salt thereof and TAK-733 or a salt thereof are administered together in the same or separate compositions. In some embodiments, osimertinib or a salt thereof and palbociclib or a salt thereof are administered together in the same or separate compositions. In some embodiments, TAK-733 or a salt thereof and palbociclib or a salt thereof are administered together in the same or separate compositions.

In another aspect, the method comprises administering an effective amount of cetuximab, TAK-733 or a salt thereof, and palbociclib or a salt thereof. In some embodiments, cetuximab is administered before TAK-733 or a salt thereof and palbociclib or a salt thereof. In some embodiments, TAK-733 or a salt thereof is administered before cetuximab and palbociclib or a salt thereof. In some embodiments, palbociclib or a salt thereof is administered before cetuximab and TAK-733 or a salt thereof. In some embodiments, cetuximab and TAK-733 or a salt thereof are administered together in the same or separate compositions. In some embodiments, cetuximab and palbociclib or a salt thereof are administered together in the same or separate compositions. In some embodiments, TAK-733 or a salt thereof and palbociclib or a salt thereof are administered together in the same or separate compositions. In some embodiments, cetuximab is administered in a separate composition from TAK-733 or a salt thereof, and palbociclib or a salt thereof. In some embodiments, cetuximab is administered every week or every two weeks, while TAK-733 or a salt thereof and palbociclib or a salt thereof are administered on a daily basis.

In another aspect, the method comprises administering an effective amount of cetuximab, trametinib or a salt thereof, and palbociclib or a salt thereof. In some embodiments, cetuximab is administered before trametinib or a salt thereof and palbociclib or a salt thereof. In some embodiments, trametinib or a salt thereof is administered before cetuximab and palbociclib or a salt thereof. In some embodiments, palbociclib or a salt thereof is administered before cetuximab and trametinib or a salt thereof. In some embodiments, cetuximab and trametinib or a salt thereof are administered together in the same or separate compositions. In some embodiments, cetuximab and palbociclib or a salt thereof are administered together in the same or separate compositions. In some embodiments, trametinib or a salt thereof and palbociclib or a salt thereof are administered together in the same or separate compositions. In some embodiments, cetuximab is administered in a separate composition from trametinib or a salt thereof and palbociclib or a salt thereof. In some embodiments, cetuximab is administered every week or every two weeks, while trametinib or a salt thereof and palbociclib or a salt thereof are administered on a daily basis.

In another aspect, the method comprises administering an effective amount of avitinib or a salt thereof, cobimetinib or a salt thereof, and palbociclib or a salt thereof. In some embodiments, avitinib or a salt thereof is administered before cobimetinib or a salt thereof and palbociclib or a salt thereof. In some embodiments, cobimetinib or a salt thereof is administered before avitinib or a salt thereof and palbociclib or a salt thereof. In some embodiments, palbociclib or a salt thereof is administered before avitinib or a salt thereof and cobimetinib or a salt thereof. In some embodiments, avitinib or a salt thereof and cobimetinib or a salt thereof are administered together in the same or separate compositions. In some embodiments, avitinib or a salt thereof and palbociclib or a salt thereof are administered together in the same or separate compositions. In some embodiments, cobimetinib or a salt thereof and palbociclib or a salt thereof are administered together in the same or separate compositions. In some embodiments, avitinib or a salt thereof is administered in a separate composition from cobimetinib or a salt thereof, and palbociclib or a salt thereof. In some embodiments, avitinib or a salt thereof is administered daily, while cobimetinib or a salt thereof and palbociclib or a salt thereof are administered on a daily basis.

In another aspect, the method comprises administering an effective amount of cetuximab, binimetinib or a salt thereof, and palbociclib or a salt thereof. In some embodiments, cetuximab is administered before binimetinib or a salt thereof and palbociclib or a salt thereof. In some embodiments, binimetinib or a salt thereof is administered before cetuximab and palbociclib or a salt thereof. In some embodiments, palbociclib or a salt thereof is administered before cetuximab and binimetinib or a salt thereof. In some embodiments, cetuximab and binimetinib or a salt thereof are administered together in the same or separate compositions. In some embodiments, cetuximab and palbociclib or a salt thereof are administered together in the same or separate compositions. In some embodiments, binimetinib or a salt thereof and palbociclib or a salt thereof are administered together in the same or separate compositions. In some embodiments, cetuximab is administered in a separate composition from binimetinib or a salt thereof, and palbociclib or a salt thereof. In some embodiments, cetuximab is administered every week or every two weeks, while binimetinib or a salt thereof and palbociclib or a salt thereof are administered on a daily basis.

In another aspect, the method comprises administering an effective amount of osimertinib or a salt thereof, binimetinib or a salt thereof, and palbociclib or a salt thereof. In some embodiments, osimertinib or a salt thereof is administered before binimetinib or a salt thereof and palbociclib or a salt thereof. In some embodiments, binimetinib or a salt thereof is administered before osimertinib or a salt thereof and palbociclib or a salt thereof. In some embodiments, palbociclib or a salt thereof is administered before osimertinib or a salt thereof and binimetinib or a salt thereof. In some embodiments, osimertinib or a salt thereof and binimetinib or a salt thereof are administered together in the same or separate compositions. In some embodiments, osimertinib or a salt thereof and palbociclib or a salt thereof are administered together in the same or separate compositions. In some embodiments, binimetinib or a salt thereof and palbociclib or a salt thereof are administered together in the same or separate compositions. In some embodiments, osimertinib or a salt thereof is administered in a separate composition from binimetinib or a salt thereof, and palbociclib or a salt thereof. In some embodiments, osimertinib or a salt thereof is administered daily, while binimetinib or a salt thereof and palbociclib or a salt thereof are administered on a daily basis.

In another aspect, the method comprises administering an effective amount of cetuximab, cobimetinib or a salt thereof, and abemaciclib or a salt thereof. In some embodiments, cetuximab is administered before cobimetinib or a salt thereof and abemaciclib or a salt thereof. In some embodiments, cobimetinib or a salt thereof is administered before cetuximab and abemaciclib or a salt thereof. In some embodiments, abemaciclib or a salt thereof is administered before cetuximab and cobimetinib or a salt thereof. In some embodiments, cetuximab and cobimetinib or a salt thereof are administered together in the same or separate compositions. In some embodiments, cetuximab and abemaciclib or a salt thereof are administered together in the same or separate compositions. In some embodiments, cobimetinib or a salt thereof and abemaciclib or a salt thereof are administered together in the same or separate compositions. In some embodiments, cetuximab is administered in a separate composition from cobimetinib or a salt thereof, and abemaciclib or a salt thereof. In some embodiments, cetuximab is administered every week or every two weeks, while cobimetinib or a salt thereof and abemaciclib or a salt thereof are administered on a daily basis.

In another aspect, the method comprises administering an effective amount of osimertinib or a salt thereof, trametinib or a salt thereof, and palbociclib or a salt thereof. In some embodiments, osimertinib or a salt thereof is administered before trametinib or a salt thereof and palbociclib or a salt thereof. In some embodiments, trametinib or a salt thereof is administered before osimertinib or a salt thereof and palbociclib or a salt thereof. In some embodiments, palbociclib or a salt thereof is administered before osimertinib or a salt thereof and trametinib or a salt thereof. In some embodiments, osimertinib or a salt thereof and trametinib or a salt thereof are administered together in the same or separate compositions. In some embodiments, osimertinib or a salt thereof and palbociclib or a salt thereof are administered together in the same or separate compositions. In some embodiments, trametinib or a salt thereof and palbociclib or a salt thereof are administered together in the same or separate compositions.

In some embodiments, intermittent administrations are about 1-30 minutes apart, about 30-60 minutes apart, about 60-120 minutes apart, about 120-240 minutes apart, about 240-480 minutes apart, about 480-720 minutes apart, about 720-960 minutes apart or about 960-1440 minutes apart. In some embodiments, intermittent administrations are about 1-2 days apart, 2-3 days apart, 3-4 days apart, 4-5 days apart, 5-6 days apart, or 6-7 days apart.

In another aspect, the subject has been previously treated with a KRAS inhibitor. In another aspect, the subject has not been previously treated with a KRAS inhibitor. In another aspect, the subject has been previously treated with a combination of KRAS inhibitor and a second active agent. In another aspect, the subject has not been previously treated with a combination of KRAS inhibitor and a second active agent. In some embodiments, the subject has developed acquired or adaptive resistance to a KRAS inhibitor. KRAS inhibitors include, without limitation, a small molecule or antibody that specifically binds to wild or mutated KRAS or a ligand thereof, such as AMG-510 (“Amgen”; pyrido(2,3-d)pyrimidin-2(1H)-one, 6-fluoro-7-(2-fluoro-6-hydroxyphenyl)-1-(4-methyl-2-(1-methylethyl)-3-pyridinyl)-4-((2S)-1-oxo-2-propen-1-yl)-1-piperazinyl)), DCAI (2-(4,6-dichloro-2-methyl-1h-indol-3-yl)ethanamine,4,6-Dichloro-2-methyl-3-aminoethylindole), MRTX849 and ARS-1620.

In another aspect, the method described herein reduces cancer cell growth and/or increase cancer cell-killing by at least about 5%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, 1000%, 2000% or more than administration of only one or two of (a) an epidermal growth factor receptor (EGFR) inhibitor (e.g., osimertinib, avitinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib). And the method described herein has demonstrated a synergistic effect upon treatment of cancer with a KRAS mutation. In some embodiments, the efficacy of the method described herein is at least about 5%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, 1000%, 2000% or more than the additive efficacy of the individual administration of (a) an epidermal growth factor receptor (EGFR) inhibitor (e.g., osimertinib, avitinib, or cetuximab); (b) a mitogen-activated protein kinase (MEK) 1/2 inhibitor (e.g., cobimetinib, trametinib, binimetinib, or TAK-733); or (c) a cyclin dependent kinase (CDK) 4/6 inhibitor (e.g., palbociclib or abemaciclib).

In another aspect, the method described herein reduces mean tumor volume by about 20-95%. In some embodiments, the mean tumor volume is reduced by at least about 10%, 15%, 20%, 25%, 30%, 35%, 40%, 45%, 50%, 55%, 60%, 65%, 70%, 75%, 80%, 85%, 90%, or 95%. In some embodiments, the method described herein reduces mean tumor volume in a subject by about 20-95%. In some embodiments, the mean tumor volume in a subject is reduced by at least about 10%, 15%, 20%, 25%, 30%, 35%, 40%, 45%, 50%, 55%, 60%, 65%, 70%, 75%, 80%4, 85%, 90%, or 95%.

In another aspect, the method described herein causes body weight changes in the subject at less than about 25%, less than about 20%, less than about 15%, or less than about 5%. In some embodiments, the method does not cause body weight change.

In another aspect, the method described herein has a Maximum Inhibition Index (MI) value of at least about 10, at least about 25, at least about 50, at least about 75, at least about 100, at least about 150, at least about 200, at least about 500, at least about 750, at least about 1000, at least about 2000, at least about 3000, at least about 4000, at least about 5000, or at least about 7500.

In yet another aspect, the method described herein comprises administration of a loading dose of the combination of (a) an epidermal growth factor receptor (EGFR) inhibitor (e.g., osimertinib, avitinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib) followed by multiple separate maintenance doses of the combination. In some embodiments, each loading dose of the three inhibitors is higher than each maintenance doses. In some embodiments, the method described herein provides lower dosages, safety and/or tolerability for long-term administrations and/or treatments.

### III. COMPOSITIONS

Also provided herein are compositions comprising (a) an epidermal growth factor receptor (EGFR) inhibitor (e.g., osimertinib, avitinib or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib); wherein the composition does not comprises a KRAS inhibitor.

Also provided herein is a composition comprising osimertinib or a salt thereof, cobimetinib or a salt thereof, and palbociclib or a salt thereof. Also provided herein is a composition comprising cetuximab, cobimetinib or a salt thereof, and palbociclib or a salt thereof. Also provided herein is a composition comprising osimertinib or a salt thereof, TAK-733 or a salt thereof, and palbociclib or a salt thereof. Also provided herein is a composition comprising cetuximab or a salt thereof, TAK-733 or a salt thereof, and palbociclib or a salt thereof. Also provided herein is a composition comprising avitinib or a salt thereof, cobimetinib or a salt thereof, and palbociclib or a salt thereof. Also provided herein is a composition comprising osimertinib or a salt thereof, binimetinib or a salt thereof, and palbociclib or a salt thereof. Also provided herein is a composition comprising cetuximab, binimetinib or a salt thereof, and palbociclib or a salt thereof. Also provided herein is a composition comprising cetuximab, cobimetinib or a salt thereof, and abemaciclib or a salt thereof. Also provided herein is a composition comprising osimertinib or a salt thereof, trametinib or a salt thereof, and palbociclib or a salt thereof. Also provided herein is a composition comprising cetuximab, cobimetinib or a salt thereof, and abemaciclib or a salt thereof. Also provided herein is a composition comprising cetuximab, trametinib or a salt thereof, and palbociclib or a salt thereof These compositions may be used for treating and delaying progression of cancer with a KRAS mutation in a method described herein. In some embodiments, the composition does not comprise a KRAS inhibitor.

In one aspect, the composition may further comprise a pharmaceutically acceptable carrier, excipient, binder, or diluent. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In some embodiments, pharmaceutical compositions according to the embodiments are sterile compositions. Pharmaceutical compositions may be prepared using compounding techniques known or that become available to those skilled in the art. Sterile compositions are also contemplated by the embodiments, including compositions that are in accord with national and local regulations governing such compositions.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions provided herein may be administered by a suitable route of delivery, such as oral, parenteral, rectal, nasal, or topical route, or by inhalation. In some embodiments, the compositions are formulated for intravenous or oral administration.

In another aspect, the composition is formulated for oral administration. For oral administration, composition may be formulated in a solid form, such as a tablet or capsule, or as a solution, emulsion, or suspension. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

In another aspect, the composition comprises osimertinib or a salt thereof, cobimetinib or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60% %, or greater than about 65%, or greater than about 70% by weight.

In some embodiments, the composition comprises cobimetinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%/o, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, cobimetinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, cobimetinib or a salt thereof, and palbociclib or a salt thereof by weight in the compositions is about 2:1:4. In some embodiments, the ratio of osimertinib or a salt thereof and cobimetinib or a salt thereof by weight is in the range of about 2:3 to 4:1. In some embodiments, the ratio of osimertinib or a salt thereof and palbociclib or a salt thereof by weight is in the range of about 40:125 to 80:75. In some embodiments, the ratio of cobimetinib or a salt thereof and palbociclib or a salt thereof by weight is in the range of about 20:125 to 60:75.

In another aspect, the composition comprises osimertinib or a salt thereof, TAK-733 or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises TAK-733 or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, TAK-733 or a salt thereof and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof and TAK-733 or a salt thereof by weight in the compositions is in the range of about 5:2 to 10:1. In some embodiments, the ratio of osimertinib or a salt thereof and palbociclib or a salt thereof by weight in the compositions is in the range of about 40:125 to 80:75. In some embodiments, the ratio of TAK-733 or a salt thereof and palbociclib or a salt thereof by weight in the compositions is in the range of about 8:125 to 16:75.

In another aspect, the composition comprises osimertinib or a salt thereof, binimetinib or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises binimetinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, binimetinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, binimetinib or a salt thereof, and palbociclib or a salt thereof by weight in the compositions is about 2:1:4. In some embodiments, the ratio of osimertinib or a salt thereof and binimetinib or a salt thereof by weight is in the range of about 2:3 to 4:1. In some embodiments, the ratio of osimertinib or a salt thereof and palbociclib or a salt thereof by weight is in the range of about 40:125 to 80:75. In some embodiments, the ratio of binimetinib or a salt thereof and palbociclib or a salt thereof by weight is in the range of about 20:125 to 60:75.

In another aspect, the composition comprises avitinib or a salt thereof, cobimetinib or a salt thereof, and palbociclib or a salt thereof. In some embodiments, the composition comprises avitinib or a salt thereof at greater than about 20%, or greater than about 25%, or greater than about 30%%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises cobimetinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of avitinib or a salt thereof, cobimetinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of avitinib or a salt thereof, cobimetinib or a salt thereof, and palbociclib or a salt thereof by weight in the compositions is about 2:1:4. In some embodiments, the ratio of avitinib or a salt thereof and cobimetinib or a salt thereof by weight is in the range of about 2:3 to 4:1. In some embodiments, the ratio of avitinib or a salt thereof and palbociclib or a salt thereof by weight is in the range of about 40:125 to 80:75. In some embodiments, the ratio of cobimetinib or a salt thereof and palbociclib or a salt thereof by weight is in the range of about 20:125 to 60:75.

In another aspect, the composition comprises salts of osimertinib, cobimetinib, TAK-733 or palbociclib. In some embodiments, the salts are pharmaceutically acceptable salts. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, mesylates phenylpropionates, phenylbutyrates, citrates, lactates, γ-hydroxybutyrates, glycolates, tartrates, and mandelates. In some embodiments, the composition comprises mesylate salts of osimertinib, cobimetinib, TAK-733 or palbociclib. In some embodiments, the composition comprises fumarate salts of osimertinib, cobimetinib, TAK-733 or palbociclib. In some embodiments, the composition comprises a solvate of osimertinib, cobimetinib, TAK-733 or palbociclib.

### IV. KITS

Also provided herein are kits comprising (a) an epidermal growth factor receptor (EGFR) inhibitor (e.g., osimertinib, avitinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib); wherein the kit does not comprises a KRAS inhibitor. The kits may be used for treating and delaying progression of cancer with a KRAS mutation in a method described herein. The kit may comprise any compositions described herein.

Also provided herein is a kit comprising osimertinib or a salt thereof, cobimetinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, cobimetinib or a salt thereof, and palbociclib or a salt thereof are formulated as one composition in the kit. In some embodiments, osimertinib or a salt thereof, cobimetinib or a salt thereof, and palbociclib or a salt thereof are formulated separately. In some embodiments, osimertinib or a salt thereof, cobimetinib or a salt thereof, and/or palbociclib or a salt thereof are formulated for oral administration. In some embodiments, osimertinib or a salt thereof, cobimetinib or a salt thereof, and palbociclib or a salt thereof are formulated in the same form, such as solid or liquid form. In some embodiments, osimertinib or a salt thereof, cobimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, cobimetinib or a salt thereof, and palbociclib or a salt thereof by weight provided in the kits are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, cobimetinib or a salt thereof, and palbociclib or a salt thereof by weight in the kits is about 2:1:4. In some embodiments, the ratio of osimertinib or a salt thereof and cobimetinib or a salt thereof by weight in the kits is in the range of about 2:3 to 4:1. In some embodiments, the ratio of osimertinib or a salt thereof and palbociclib or a salt thereof by weight in the kits is in the range of about 40:125 to 80:75. In some embodiments, the ratio of cobimetinib or a salt thereof and palbociclib or a salt thereof by weight in the kits is in the range of about 20:125 to 60:75.

Also provided herein is a kit comprising cetuximab, cobimetinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, cobimetinib or a salt thereof, and palbociclib or a salt thereof are formulated as one composition in the kit. In some embodiments, cetuximab, cobimetinib or a salt thereof, and palbociclib or a salt thereof are formulated as two or more compositions (e.g., two or three compositions) in the kit. In some embodiments, cetuximab, cobimetinib or a salt thereof, and palbociclib or a salt thereof are formulated separately. In some embodiments, cobimetinib or a salt thereof, and/or palbociclib or a salt thereof are formulated for oral administration. In some embodiments, cobimetinib or a salt thereof, and palbociclib or a salt thereof are formulated in the same form, such as solid or liquid form. In some embodiments, cetuximab, cobimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, cobimetinib or a salt thereof and palbociclib or a salt thereof are formulated as one composition for oral administration.

In some embodiments, the ratios of cetuximab, cobimetinib or a salt thereof, and palbociclib or a salt thereof by weight provided in the kits are about 1:1:1, 2:1:1, 3:1:1, 4:1:1, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, cobimetinib or a salt thereof, and palbociclib or a salt thereof by weight in the kits is about 20:3:6. In some embodiments, the ratio of cetuximab and cobimetinib or a salt thereof by weight in the kits is the range of about 85:60 to 85:2. In some embodiments, the ratio of cetuximab and palbociclib or a salt thereof by weight in the kits is the range of about 17:25 to 34:3. In some embodiments, the ratio of cobimetinib or a salt thereof and palbociclib or a salt thereof by weight in the kits is the range of about 20:125 to 60:75.

Also provided herein is a kit comprising cetuximab, TAK-733 or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, TAK-733 or a salt thereof, and palbociclib or a salt thereof are formulated as one composition in the kit. In some embodiments, cetuximab, TAK-733 or a salt thereof and palbociclib or a salt thereof are formulated as two or more compositions (e.g., two or three compositions) in the kit. In some embodiments, cetuximab, TAK-733 or a salt thereof and palbociclib or a salt thereof are formulated separately. In some embodiments, TAK-733 or a salt thereof and/or palbociclib or a salt thereof are formulated for oral administration. In some embodiments, TAK-733 or a salt thereof and palbociclib or a salt thereof are formulated in the same form, such as solid or liquid form. In some embodiments, cetuximab, TAK-733 or a salt thereof and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, TAK-733 or a salt thereof and palbociclib or a salt thereof are formulated as one composition for oral administration.

In some embodiments, the ratios of cetuximab, TAK-733 or a salt thereof and palbociclib or a salt thereof by weight provided in the kits are about 5:1:5, 10:1:5, 15:1:5, 20:1:5, 10:2:1, 10:1:2, 10:1:3, 10:1:4, 10:1:5, 10:1:6, 10:1:7, 10:1:8, 5:1:9, 10:1:10, 15:1:2, 15:1:3, 15:1:4, 15:1:5, 15:1:6, 15:1:7, 15:1:8, 15:1:9, 15:1:10, 20:1:1, 20:1:2, 20:1:3, 20:1:4, 20:1:5, 20:1:6, 20:1:7, 20:1:8, 20:1:9, or 20:1:10. In some embodiments, the ratio of cetuximab, TAK-733 or a salt thereof and palbociclib or a salt thereof by weight in the kits is about 20:1:6. In some embodiments, the ratio of cetuximab and TAK-733 or a salt thereof by weight in the kits is in the range of about 85:16 to 850:8. In some embodiments, the ratio of cetuximab and palbociclib or a salt thereof by weight in the kits is in the range of about 17:25 to 34:3. In some embodiments, the ratio of TAK-733 or a salt thereof and palbociclib or a salt thereof by weight in the kits is in the range of about 8:125 to 16:75.

Also provided herein is a kit comprising osimertinib or a salt thereof, TAK-733 or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, TAK-733 or a salt thereof, and palbociclib or a salt thereof are formulated as one composition in the kit. In some embodiments, osimertinib or a salt thereof, TAK-733 or a salt thereof, and palbociclib or a salt thereof are formulated separately. In some embodiments, osimertinib or a salt thereof, TAK-733 or a salt thereof, and/or palbociclib or a salt thereof are formulated for oral administration. In some embodiments, osimertinib or a salt thereof, TAK-733 or a salt thereof, and palbociclib or a salt thereof are formulated in the same form, such as solid or liquid form. In some embodiments, osimertinib or a salt thereof, TAK-733 or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, TAK-733 or a salt thereof, and palbociclib or a salt thereof by weight provided in the kits are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, TAK-733 or a salt thereof and palbociclib or a salt thereof by weight in the compositions is about 3:1:6. In some embodiments, the ratio of osimertinib or a salt thereof and TAK-733 or a salt thereof by weight in the kits is in the range of about 5:2 to 10:1. In some embodiments, the ratio of osimertinib or a salt thereof and palbociclib or a salt thereof by weight in the kits is in the range of about 40:125 to 80:75. In some embodiments, the ratio of TAK-733 or a salt thereof and palbociclib or a salt thereof by weight in the kits is in the range of about 8:125 to 16:75.

Also provided herein is a kit comprising cetuximab, trametinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, trametinib or a salt thereof, and palbociclib or a salt thereof are formulated as one composition in the kit. In some embodiments, cetuximab, trametinib or a salt thereof and palbociclib or a salt thereof are formulated as two or more compositions (e.g., two or three compositions) in the kit. In some embodiments, cetuximab, trametinib or a salt thereof and palbociclib or a salt thereof are formulated separately. In some embodiments, trametinib or a salt thereof and/or palbociclib or a salt thereof are formulated for oral administration. In some embodiments, trametinib or a salt thereof and palbociclib or a salt thereof are formulated in the same form, such as solid or liquid form. In some embodiments, cetuximab, trametinib or a salt thereof and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, trametinib or a salt thereof and palbociclib or a salt thereof are formulated as one composition for oral administration.

Also provided herein is a kit comprising osimertinib or a salt thereof, trametinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, trametinib or a salt thereof, and palbociclib or a salt thereof are formulated as one composition in the kit. In some embodiments, osimertinib or a salt thereof, trametinib or a salt thereof, and palbociclib or a salt thereof are formulated separately. In some embodiments, osimertinib or a salt thereof, trametinib or a salt thereof, and/or palbociclib or a salt thereof are formulated for oral administration. In some embodiments, osimertinib or a salt thereof, trametinib or a salt thereof, and palbociclib or a salt thereof are formulated in the same form, such as solid or liquid form. In some embodiments, osimertinib or a salt thereof, trametinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, trametinib or a salt thereof, and palbociclib or a salt thereof by weight provided in the kits are about 15:1:10, 20:1:10, 25:1:10, 30:1:10, 35:1:10, 40:1:10, 50:1:10, 15:1:20, 20:1:20, 25:1:20, 30:1:20, 35:1:20, 40:1:20, 50:1:20, 15:1:30, 20:1:30, 25:1:30, 30:1:30, 35:1:30, 40:1:30, 50:1:30 or 40:1:50. In some embodiments, the ratio of osimertinib or a salt thereof, trametinib or a salt thereof, and palbociclib or a salt thereof by weight in the compositions is about 15:1:12. In some embodiments, the ratio of osimertinib or a salt thereof and trametinib or a salt thereof by weight in the kits is the range of about 20:1 to 160:1. In some embodiments, the ratio of osimertinib or a salt thereof and palbociclib or a salt thereof by weight in the kits is the range of about 40:125 to 80:75. In some embodiments, the ratio of trametinib or a salt thereof and palbociclib or a salt thereof by weight in the kits is the range of about 1:250 to 2:75.

Also provided herein is a kit comprising cetuximab, binimetinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising binimetinib or a salt thereof, and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, binimetinib or a salt thereof, and palbociclib or a salt thereof are formulated as two or more compositions in the kit. In some embodiments, cetuximab, binimetinib or a salt thereof, and palbociclib or a salt thereof are formulated separately. In some embodiments, binimetinib or a salt thereof, and/or palbociclib or a salt thereof are formulated for oral administration. In some embodiments, binimetinib or a salt thereof, and palbociclib or a salt thereof are formulated in the same form, such as solid or liquid form. In some embodiments, cetuximab, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, binimetinib or a salt thereof and palbociclib or a salt thereof are formulated as one composition for oral administration.

In some embodiments, the ratios of cetuximab, binimetinib or a salt thereof, and palbociclib or a salt thereof by weight provided in the kits are about 1:1:1, 2:1:1, 3:1:1, 4:1:1, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, binimetinib or a salt thereof, and palbociclib or a salt thereof by weight in the kits is about 20:3:6. In some embodiments, the ratio of cetuximab and binimetinib or a salt thereof by weight in the kits is the range of about 85:60 to 85:2. In some embodiments, the ratio of cetuximab and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising osimertinib or a salt thereof, binimetinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, binimetinib or a salt thereof, and palbociclib or a salt thereof are formulated as one composition in the kit. In some embodiments, osimertinib or a salt thereof, binimetinib or a salt thereof, and palbociclib or a salt thereof are formulated as two or more compositions in the kit. In some embodiments, osimertinib or a salt thereof, binimetinib or a salt thereof, and palbociclib or a salt thereof are formulated separately. In some embodiments, osimertinib or a salt thereof, binimetinib or a salt thereof, and/or palbociclib or a salt thereof are formulated for oral administration. In some embodiments, osimertinib or a salt thereof, binimetinib or a salt thereof, and palbociclib or a salt thereof are formulated in the same form, such as solid or liquid form. In some embodiments, osimertinib or a salt thereof, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, osimertinib or a salt thereof, binimetinib or a salt thereof and palbociclib or a salt thereof are formulated as one composition for oral administration.

In some embodiments, the ratios of osimertinib or a salt thereof, binimetinib or a salt thereof, and palbociclib or a salt thereof by weight provided in the kits are about 1:1:1, 2:1:1, 3:1:1, 4:1:1, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 51:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of osimertinib or a salt thereof, binimetinib or a salt thereof, and palbociclib or a salt thereof by weight in the kits is about 20:3:6. In some embodiments, the ratio of osimertinib or a salt thereof and binimetinib or a salt thereof by weight in the kits is the range of about 85:60 to 85:2. In some embodiments, the ratio of osimertinib or a salt thereof and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising cetuximab, cobimetinib or a salt thereof, and abemaciclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising abemaciclib or a salt thereof and a pharmaceutically acceptable carrier, excipient, binder, or diluent.

In some embodiments, cetuximab, cobimetinib or a salt thereof, and abemaciclib or a salt thereof are formulated as one composition in the kit. In some embodiments, cetuximab, cobimetinib or a salt thereof, and abemaciclib or a salt thereof are formulated as two or more compositions in the kit. In some embodiments, cetuximab, cobimetinib or a salt thereof, and abemaciclib or a salt thereof are formulated separately. In some embodiments, cobimetinib or a salt thereof, and/or abemaciclib or a salt thereof are formulated for oral administration. In some embodiments, cobimetinib or a salt thereof, and abemaciclib or a salt thereof are formulated in the same form, such as solid or liquid form. In some embodiments, cetuximab, cobimetinib or a salt thereof, and/or abemaciclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, cobimetinib or a salt thereof and abemaciclib or a salt thereof are formulated as one composition for oral administration.

In some embodiments, the ratios of cetuximab, cobimetinib or a salt thereof, and abemaciclib or a salt thereof by weight provided in the kits are about 1:1:1, 2:1:1, 3:1:1, 4:1:1, 1:2:1, 3:1:2, 3:1:3, 3:1:4, 3:1:5, 3:1:6, 3:1:7, 3:1:8, 3:1:9, 3:1:10, 4:1:2, 4:1:3, 41:4, 4:1:5, 4:1:6, 4:1:7, 4:1:8, 4:1:9, 4:1:10, 5:1:1, 5:1:2, 5:1:3, 51:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, cobimetinib or a salt thereof, and abemaciclib or a salt thereof by weight in the kits is about 20:3:6. In some embodiments, the ratio of cetuximab and cobimetinib or a salt thereof by weight in the kits is the range of about 85:60 to 85:2. In some embodiments, the ratio of cetuximab and abemaciclib or a salt thereof by weight in the kits is the range of about 17:25 to 34:3. In some embodiments, the ratio of cobimetinib or a salt thereof and abemaciclib or a salt thereof by weight in the kits is the range of about 20:125 to 60:75.

Also provided herein is a kit comprising avitinib or a salt thereof, cobimetinib or a salt thereof, and palbociclib or a salt thereof. In some embodiments, the kit comprises a pharmaceutical composition comprising avitinib or a salt thereof and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, avitinib or a salt thereof, cobimetinib or a salt thereof, and palbociclib or a salt thereof are formulated as one composition in the kit. In some embodiments, avitinib or a salt thereof, cobimetinib or a salt thereof, and palbociclib or a salt thereof are formulated as two or more compositions in the kit. In some embodiments, avitinib or a salt thereof, cobimetinib or a salt thereof, and palbociclib or a salt thereof are formulated separately. In some embodiments, avitinib or a salt thereof, cobimetinib or a salt thereof, and/or palbociclib or a salt thereof are formulated for oral administration. In some embodiments, cobimetinib or a salt thereof, and palbociclib or a salt thereof are formulated in the same form, such as solid or liquid form. In some embodiments, avitinib or a salt thereof, cobimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, avitinib or a salt thereof, cobimetinib or a salt thereof and palbociclib or a salt thereof are formulated as one composition for oral administration.

In some embodiments, the ratios of avitinib or a salt thereof, cobimetinib or a salt thereof, and palbociclib or a salt thereof by weight provided in the kits are about 1:1:1, 2:1:1, 3:1:1, 4:1:1, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of avitinib or a salt thereof, cobimetinib or a salt thereof, and palbociclib or a salt thereof by weight in the kits is about 20:3:6. In some embodiments, the ratio of avitinib or a salt thereof and cobimetinib or a salt thereof by weight in the kits is the range of about 85:60 to 85:2. In some embodiments, the ratio of avitinib or a salt thereof and palbociclib or a salt thereof by weight in the kits is the range of about 17:25 to 34:3. In some embodiments, the ratio of cobimetinib or a salt thereof and palbociclib or a salt thereof by weight in the kits is the range of about 20:125 to 60:75.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments, the kits may include a dosage amount of at least one composition as disclosed herein.

### EXAMPLES

The following examples are offered to illustrate but not to limit the invention. One of skill in the art will recognize that the following procedures may be modified using methods known to one of ordinary skill in the art.

### Example 1: Ex Vivo Test of Maximum Inhibition Index (MI) and Growth Inhibition for NSCLC Cell Lines with KRAS Mutations Treated with Solo and Combination Therapies

For ex vivo drug testing, the cells were exposed to a combination of drugs or single drugs for 4 days or 7 days. Following exposure with drug, 1,000-5,000 cells were labeled with 5-ethynyl-2′-deoxyuridine (Edu) to assess the tumor cell proliferation rates. The labeling lasted 24 hours in the presence of drug exposure. In the control group, epithelial tumor cells received no drug exposure with media change (with 0.1% DMSO), but were similarly labeled with Edu. The labeled cells were fixed and blocked with 3% BSA and 0.5% Triton X-100 for 2 hours at room temperature, followed by staining with Hoechst 33342 and EpCAM antibody (1:4000) in PBS solution containing 0.5% Triton X-100 overnight at 4° C. Subsequently, the cells were rinsed with PBST then incubated with Alexa Fluor® 647 conjugated goat-antimouse secondary antibody for 30 minutes at room temperature.

The incorporated Edu was detected by Click-iT reaction where fixed cells were incubated with a reaction mixture containing 1× Click-iT Edu reaction buffer, CuSO₄, and azide-conjugated Alexa Fluor dye in the dark. The stained cells were washed with PBS two times before image acquisition and analysis.

For image acquisition and analysis, the stained tumor cells were imaged by a high-content screening (HCS) platform (Thermo Scientific CellomicsArrayScanXTi HCS reader). The 10× objective was used to collect images. Twenty-five fields were imaged for each well for the analysis. From the images three fluorescent signals were obtained from the HCS reader. Blue fluorescent signals recorded nucleus signals stained with Hoechst 33342, green fluorescent signal detected the Edu incorporated in newly synthesized DNA, and red fluorescent signal detected the EpCAM positive epithelial cells population.

The MI (Maximum Inhibition Index) was calculated using the EpCAM and Edu positive readout (Tables 1-4). MI=Edu positive cells in control/Edu positive cells in treatment. MI4 indicates an MI value after 4 days of treatment and MI7 indicates an MI values after 7 days of treatment. The MI and growth inhibition rate (GI %) values for KRAS^{G12A}-NSCLC (H1573 and H2009), KRAS^{G12C}-NSCLC (H358, H23 and H2030), KRAS^{G12V}-NSCLC (H2291 and H2444) and KRAS^{G12D}-NSCLC (H1588 and SK-LU-1) cell lines are shown in Tables 1-4, respectively. GI % is calculated as GI %=(1−1/MI)*100%.

TABLE 1
Maximum Inhibition Index (MI) and Growth Inhibition Rate (GI %) during Solo
and Combination Therapies for KRAS^{G12A}-NSCLC (H1573 and H2009 cell lines)

| | Concentration | H1573 | | H2009 | |
|---|---|---|---|---|---|
| Samples | (uM) | GI % | MI 4 | GI % | MI 4 |
| Osimertinib + Cobimetinib + | 0.5 + 0.4 + 0.15 | 99.92 | 1178.75 | 79.71 | 4.93 |
| Palbociclib | | | | | |
| Osimertinib + Cobimetinib + | 0.5 + 0.4 + 5 | 99.98 | 6419.86 | 78.83 | 4.72 |
| Ribociclib | | | | | |
| Osimertinib + Trametinib + Palbociclib | 0.5 + 0.02 + 0.15 | 99.95 | 2124.09 | 72.78 | 3.67 |
| Osimertinib + Trametinib + Ribociclib | 0.5 + 0.02 + 5 | 99.99 | 13140.64 | 73.48 | 3.77 |
| Afatinib + Tramctinib + Palbociclib | 0.2 + 0.02 + 0.15 | 99.49 | 197.17 | 63.62 | 2.75 |
| Afatinib + Trametinib + Ribociclib | 0.2 + 0.02 + 5 | 99.96 | 2624.85 | 65.47 | 2.90 |
| Afatinib + TAK-733 + Ribociclib | 0.2 + 0.18 + 5 | 99.93 | 1440.17 | 69.00 | 3.23 |
| Afatinib + TAK-733 + Palbociclib | 0.2 + 0.18 + 0.15 | 99.94 | 1637.61 | 69.78 | 3.31 |
| Osimertinib + TAK-733 + Ribociclib | 0.5 + 0.18 + 5 | 99.99 | 6723.07 | 75.36 | 4.06 |
| Osimertinib + TAK-733 + Palbociclib | 0.5 + 0.18 + 0.15 | 99.97 | 3162.73 | 74.16 | 3.87 |
| Osimertinib + TAK733 | 0.5 + 0.18 | 99.28 | 139.50 | 77.11 | 4.37 |
| Palbociclib + TAK-733 | 0.15 + 0.18 | 99.85 | 674.53 | 71.79 | 3.54 |
| Cobimetinib + Palbociclib | 0.4 + 0.15 | 99.94 | 1634.78 | 76.93 | 4.33 |
| Osimertinib + Palbociclib | 0.5 + 0.15 | 97.87 | 46.96 | 28.19 | 1.39 |
| Osimertinib + Cobimetinib | 0.5 + 0.4 | 99.63 | 272.94 | 84.16 | 6.31 |
| TAK-733 | 0.18 | 89.62 | 9.64 | 62.18 | 2.64 |
| Trametinib | 0.02 | 87.80 | 8.20 | 65.93 | 2.93 |
| Ribociclib | 5 | 99.00 | 100.43 | 10.09 | 1.11 |
| Palbociclib | 0.15 | 92.17 | 12.77 | 8.82 | 1.10 |
| Cobimetinib | 0.4 | 96.36 | 27.49 | 67.99 | 3.12 |
| Afatinib | 0.2 | 42.36 | 1.73 | 14.83 | 1.17 |
| Osimertinib | 0.5 | 67.71 | 3.10 | 35.30 | 1.55 |

TABLE 2a
Maximum Inhibition Index (MI) and Growth Inhibition Rate (GI %) during Solo
and Combination Therapies for KRAS^{G12C}-NSCLC (H358, H23 and H2030 cell lines)

| | Concentration | H358 | | H23 | | H2030 | |
|---|---|---|---|---|---|---|---|
| Samples | (uM) | GI % | MI 7 | GI % | MI 7 | GI % | MI 7 |
| Osimertinib + | 0.5 + | 99.98 | 7921.38 | 91.43 | 11.67 | 99.67 | 301.05 |
| Cobimetinib + Palbociclib | 0.4 + 0.15 | | | | | | |
| Osimertinib + | 0.5 + | 99.94 | 1609.92 | 94.09 | 17.05 | 99.51 | 201.54 |
| Cobimetinib + Ribociclib | 0.4 + 5 | | | | | | |
| Osimertinib + | 0.5 + | 94.47 | 18.09 | 63.55 | 2.77 | 97.14 | 34.98 |
| Trametinib + Palbociclib | 0.02 + 0.15 | | | | | | |
| Osimertinib + | 0.5 + | 99.98 | 4781.58 | 77.61 | 4.47 | 99.65 | 285.10 |
| Trametinib + Ribociclib | 0.02 + 5 | | | | | | |
| Afatinib + | 0.2 + | 95.82 | 23.26 | 62.79 | 2.69 | 97.79 | 45.17 |
| Trametinib + Palbociclib | 0.02 + 0.15 | | | | | | |
| Afatinib + | 0.2 + | 99.85 | 676.47 | 81.18 | 5.31 | 99.00 | 99.91 |
| Trametinib + Ribociclib | 0.02 + 5 | | | | | | |
| Trametinib | 0.02 | 20.76 | 1.26 | 1.20 | 1.01 | 64.45 | 2.81 |
| Ribociclib | 5 | 95.43 | 21.19 | 74.57 | 3.96 | 84.67 | 6.57 |
| Palbociclib | 0.15 | 60.31 | 2.53 | 41.73 | 1.72 | 52.39 | 2.10 |
| Cobimetinib | 0.4 | 97.48 | 39.68 | 75.83 | 4.25 | 92.44 | 13.21 |
| Afatinib | 0.2 | 22.84 | 1.30 | 16.09 | 0.87 | 55.33 | 2.23 |
| Osimertinib | 0.5 | 22.86 | 1.30 | 13.11 | 0.89 | 57.39 | 2.35 |
| Afatinib + | 0.2 + | 99.96 | 2428.95 | 90.95 | 11.08 | 99.13 | 116.79 |
| Cobimetinib + Palbociclib | 0.4 + 0.15 | | | | | | |
| Afatinib + | 0.2 + | 99.92 | 1320.27 | 94.45 | 18.03 | 99.46 | 184.49 |
| Cobimetinib + Ribociclib | 0.4 + 5 | | | | | | |

TABLE 2b
Maximum Inhibition Index (MI) and Growth Inhibition
Rate (GI %) during Solo and Combination Therapies for
KRAS^{G12C}-NSCLC (H358 and H23 cell lines)

| | Concentration | H358 | | H23 | |
|---|---|---|---|---|---|
| Samples | (uM) | GI % | MI 4 | GI % | MI 4 |
| Osimertinib + | 0.5 + 0.4 + 0.15 | 99.88 | 810.84 | 89.43 | 9.46 |
| Cobimetinib + | | | | | |
| Palbociclib | | | | | |

As shown in Table 2b, the triplet combination of osimertinib, cobimetinib and palbociclib, which are an EGFR inhibitor, an MEK1/2 inhibitor and a CDK4/6 inhibitor, respectively, showed significantly growth inhibition rate (GI %) for KRAS^{G12C}-NSCLC cell lines of H358 and H23

TABLE 3
Maximum Inhibition Index (MI) and Growth Inhibition Rate (GI %) during Solo
and Combination Therapies for KRAS^{G12V}-NSCLC (H2291 and H2444 cell lines)

| | Concentration | H2291 | | H2444 | |
|---|---|---|---|---|---|
| Samples | (uM) | GI % | MI 7 | GI % | MI 4 |
| Osimertinib + Trametinib + Palbociclib | 0.5 + 0.02 + 0.15 | 100.00 | 1039.79 | 99.72 | 358.81 |
| Osimertinib + Trametinib + Ribociclib | 0.5 + 0.02 + 5 | 99.86 | 712.39 | 99.62 | 264.41 |
| Afatinib + Trametinib + Palbociclib | 0.2 + 0.02 + 0.15 | 100.00 | 1039.79 | 99.66 | 296.49 |
| Afatinib + Trametinib + Ribociclib | 0.2 + 0.02 + 5 | 99.95 | 2157.23 | 99.58 | 239.89 |
| Afatinib + TAK-733 + Ribociclib | 0.2 + 0.18 + 5 | 99.95 | 2035.80 | 99.80 | 501.25 |
| Afatinib + TAK-733 + Palbociclib | 0.2 + 0.18 + 0.15 | 99.95 | 2106.96 | 99.86 | 710.66 |
| Osimertinib + TAK-733 + Ribociclib | 0.5 + 0.18 + 5 | 100.00 | 1039.79 | 99.94 | 1703.60 |
| Osimertinib + TAK-733 + Palbociclib | 0.5 + 0.18 + 0.15 | 99.95 | 1917.01 | 99.97 | 3155.91 |
| Osimertinib + TAK 733 | 0.5 + 0.18 | 99.86 | 676.06 | 99.83 | 588.80 |
| Palbociclib + TAK-733 | 0.15 + 0.18 | 99.95 | 2347.02 | 99.63 | 269.57 |
| TAK-733 | 0.18 | 98.27 | 57.57 | 99.01 | 100.76 |
| Trametinib | 0.02 | 75.62 | 4.11 | 87.22 | 7.82 |
| Ribociclib | 5 | 98.65 | 72.98 | 74.54 | 3.93 |
| Palbociclib | 0.15 | 81.25 | 5.36 | 59.59 | 2.47 |
| Afatinib | 0.2 | 26.00 | 1.35 | 44.30 | 1.80 |
| Osimertinib | 0.5 | 21.19 | 1.30 | 66.51 | 2.99 |

TABLE 4
Maximum Inhibition Index (MI) and Growth Inhibition Rate (GI %) during Solo
and Combination Therapies for KRAS^{G12D}-NSCLC (H1588 and SK-LU-1 cell lines)

| | Concentration | H1588 | | SK-LU-1 | |
|---|---|---|---|---|---|
| Samples | (uM) | GI % | MI 4 | GI % | MI 4 |
| Osimertinib + Cobimetinib + Palbociclib | 0.5 + 0.4 + 0.15 | 98.16 | 54.32 | 96.40 | 27.76 |
| Osimertinib + Cobimetinib + Ribociclib | 0.5 + 0.4 + 5 | 99.30 | 142.32 | 98.87 | 88.56 |
| Osimertinib + Trametinib + Palbociclib | 0.5 + 0.02 + 0.15 | 97.38 | 38.14 | 96.44 | 28.12 |
| Osimertinib + Trametinib + Ribociclib | 0.5 + 0.02 + 5 | 98.86 | 87.95 | 98.64 | 73.60 |
| Afatinib + Trametinib + Palbociclib | 0.2 + 0.02 + 0.15 | 92.94 | 14.16 | 90.94 | 11.04 |
| Afatinib + Trametinib + Ribociclib | 0.2 + 0.02 + 5 | 97.70 | 43.47 | 98.17 | 54.66 |
| Afatinib + TAK-733 + Ribociclib | 0.2 + 0.18 + 5 | 98.14 | 53.67 | 97.94 | 48.49 |
| Afatinib + TAK-733 + Palbociclib | 0.2 + 0.18 + 0.15 | 96.54 | 28.91 | 95.77 | 23.64 |
| Osimertinib + TAK-733 + Ribociclib | 0.5 + 0.18 + 5 | 99.00 | 99.54 | 99.06 | 106.49 |
| Osimertinib + TAK-733 + Palbociclib | 0.5 + 0.18 + 0.15 | 98.82 | 84.48 | 96.85 | 31.74 |
| Osimertinib + TAK733 | 0.5 + 0.18 | 92.07 | 12.61 | 80.74 | 5.19 |
| Palbociclib + TAK-733 | 0.15 + 0.18 | 89.72 | 9.73 | 96.76 | 30.91 |
| Cobimetinib + Palbociclib | 0.4 + 0.15 | 92.28 | 12.95 | 96.83 | 31.52 |
| Osimertinib + Palbociclib | 0.5 + 0.15 | 85.29 | 6.80 | 81.23 | 5.33 |
| Osimertinib + Cobimetinib | 0.5 + 0.4 | 94.50 | 18.18 | 83.14 | 5.93 |
| TAK-733 | 0.18 | 36.93 | 1.59 | 77.44 | 4.43 |
| Trametinib | 0.02 | 45.63 | 1.84 | 47.99 | 1.92 |
| Ribociclib | 5 | 60.57 | 2.54 | 91.58 | 11.88 |
| Palbociclib | 0.15 | 53.52 | 2.15 | 73.45 | 3.77 |
| Cobimetinib | 0.4 | 48.38 | 1.94 | 64.82 | 2.84 |
| Afatinib | 0.2 | 43.02 | 1.75 | 8.65 | 1.09 |
| Osimertinib | 0.5 | 55.11 | 2.23 | 35.73 | 1.56 |

The triplet combination of an EGFR inhibitor such as osimertinib, an MEK 1/2 inhibitor such as cobimetinib, trametinib or TAK-733, and a CDK 4/6 inhibitor such as palbociclib or ribociclib showed significant inhibition of NSCLC cell lines with KRAS mutations of G12C and G12V, and moderate inhibition of NSCLC cell lines with KRAS mutations of G12A and G12D.

Concentration dependency of the triplet combinations were tested in KRAS^{G12A}-NSCLC (H1573), KRAS^{G12}C-NSCLC (H358 and H23), KRAS^{G12V}-NSCLC (H2444) and KRAS^{G12D}-NSCLC (H1588) cell lines at various MEK 1/2 inhibitor and CDK 4/6 inhibitor concentrations. The MI and growth inhibition rate (GI %) values are shown in tables 5 and 6.

TABLE 5
Maximum Inhibition Index (MI) and Growth Inhibition Rate (GI %) during Triplet Combination
Therapies for KRAS^{G12C}-NSCLC(H23 and H358) and KRAS^{G12A}-NSCLC (H1573)

| | Concentration | H23 | | H358 | | H1573 | |
|---|---|---|---|---|---|---|---|
| Samples | (uM) | GI % | MI 4 | GI % | MI 4 | GI % | MI 4 |
| Cetuximab + | 20 + | 87.40 | 7.94 | 99.82 | 541.43 | 99.93 | 1523.85 |
| Cobimetinib + Palbociclib | 0.4 + 0.15 | | | | | | |
| Cetuximab + | 20 + | 82.62 | 5.75 | 99.46 | 185.40 | 99.94 | 1677.38 |
| Cobimetinib + 3/5Palbociclib | 0.4 + 0.09 | | | | | | |
| Cetuximab + | 20 + | 75.99 | 4.16 | 94.62 | 18.59 | 99.57 | 231.62 |
| Cobimetinib + 1/5Palbociclib | 0.4 + 0.03 | | | | | | |
| Cetuximab + | 20 + | 79.48 | 4.87 | 99.31 | 144.48 | 99.93 | 1513.49 |
| 2/3Cobimetinib + Palbociclib | 0.267 + 0.15 | | | | | | |
| Cetuximab + | 20 + | 76.95 | 4.34 | 98.90 | 90.60 | 99.77 | 437.84 |
| 2/3Cobimetinib + 3/5Palbociclib | 0.267 + 0.09 | | | | | | |
| Cetuximab + | 20 + | 69.56 | 3.28 | 94.86 | 19.44 | 99.04 | 103.67 |
| 2/3Cobimetinib + 1/5Palbociclib | 0.267 + 0.03 | | | | | | |
| Cetuximab + | 20 + | 71.31 | 3.49 | 98.02 | 50.57 | 99.82 | 553.14 |
| 1/3Cobimetinib + Palbociclib | 0.133 + 0.15 | | | | | | |
| Cetuximab + | 20 + | 64.19 | 2.79 | 95.64 | 22.93 | 99.73 | 372.05 |
| 1/3Cobimetinib + 3/5Palbociclib | 0.133 + 0.09 | | | | | | |
| Cetuximab + | 20 + | 57.87 | 2.37 | 88.13 | 8.42 | 97.38 | 38.19 |
| 1/3Cobimetinib + 1/5Palbociclib | 0.133 + 0.03 | | | | | | |
| Osimertinib + | 0.5 + | 89.43 | 9.46 | 99.88 | 810.84 | 99.92 | 1284.56 |
| Cobimetinib + Palbociclib | 0.4 + 0.15 | | | | | | |
| Cetuximab + | 20 + | 67.92 | 3.12 | 88.62 | 8.78 | 99.54 | 219.32 |
| Binimetinib + Palbociclib | 0.2 + 0.15 | | | | | | |
| Cetuximab + | 20 + | 56.94 | 2.32 | 82.69 | 5.78 | 98.23 | 56.42 |
| Binimetinib + 3/5Palbociclib | 0.2 + 0.09 | | | | | | |
| Cetuximab + | 20 + | 38.98 | 1.64 | 64.66 | 2.83 | 92.23 | 12.87 |
| Binimetinib + 1/5Palbociclib | 0.2 + 0.03 | | | | | | |

TABLE 6
Maximum Inhibition Index (MD) and Growth Inhibition
Rate (GI %) during Triplet Combination Therapies for KRAS^{G12D}-NSCLC(H1588)
and KRAS^{G12V}-NSCLC (H2444)

| | Concentration | H1588 | | H2444 | |
|---|---|---|---|---|---|
| Samples | (uM) | GI % | MI 4 | GI % | MI 4 |
| Cetuximab + Cobimetinib + | 20 + 0.4 + 0.15 | 82.65 | 5.76 | 99.40 | 165.98 |
| Palbociclib | (full dose) | | | | |
| Cetuximab + Cobimetinib + | 20 + 0.4 + 0.09 | 75.93 | 4.15 | 99.57 | 231.17 |
| ⅗Palbociclib | | | | | |
| Cetuximab + Cobimetinib + | 20 + 0.4 + 0.03 | 63.10 | 2.71 | 99.49 | 195.31 |
| ⅕Palbociclib | | | | | |
| Cetuximab + ⅔Cobimetinib + | 20 + 0.267 + 0.15 | 78.95 | 4.75 | 99.55 | 220.36 |
| Palbociclib | | | | | |
| Cetuximab + ⅔Cobimetinib + | 20 + 0.267 + 0.09 | 74.79 | 3.97 | 99.30 | 142.73 |
| ⅗Palbociclib | | | | | |
| Cetuximab + ⅔Cobimetinib + | 20 + 0.267 + 0.03 | 57.80 | 2.37 | 99.42 | 173.89 |
| ⅕Palbociclib | | | | | |
| Cetuximab + ⅓Cobimetinib + | 20 + 0.133 + 0.15 | 77.22 | 4.39 | 98.94 | 94.22 |
| Palbociclib | | | | | |
| Cetuximab + ⅓Cobimetinib + | 20 + 0.133 + 0.09 | 69.62 | 3.29 | 98.80 | 83.18 |
| ⅗Palbociclib | | | | | |
| Cetuximab + ⅓Cobimetinib + | 20 + 0.133 + 0.03 | 53.82 | 2.17 | 97.41 | 38.66 |
| ⅕Palbociclib | | | | | |
| Osimertinib + Cobimetinib + | 0.5 + 0.4 + 0.15 | 94.49 | 18.14 | 99.93 | 1430.07 |
| Palbociclib | (full dose) | | | | |
| Cetuximab + Binimetinib + | 20 + 0.2 + 0.15 | 77.08 | 4.36 | 98.22 | 56.33 |
| Palbociclib | (full dose) | | | | |
| Cetuximab + Binimetinib + | 20 + 0.2 + 0.09 | 70.38 | 3.38 | 97.61 | 41.87 |
| ⅗Palbociclib | | | | | |
| Cetuximab + Binimetinib + | 20 + 0.2 + 0.03 | 52.23 | 2.09 | 97.12 | 34.67 |
| ⅕Palbociclib | | | | | |

As shown in Tables 5 and 6, combined inhibition of EGFR, MEK1/2 and CDK4/6 suppressed tumor cell growth across wide range of KRAS mutant NSCLC cell lines. Reduction of a single drug had less impact on the tumor inhibitory effect compared to reduction of two drugs. Dose reduction of both cobimetinib and palbociclib decreased the Growth Inhibition Rate (GI %) in H23 and H1588 by approximately 30% at ⅓ cobimetinib and ⅕ palbociclib concentrations. Remarkably, growth inhibitions of H358, H1573, and H2444 were minimally impacted at cobimetinib and palbociclib concentrations as low as ⅓ and ⅕ of the full dose, respectively. These results demonstrated that the amount of drugs used in the combination therapy can be significantly reduced without compromising clinical efficacy. Reduction of the dose may reduce drug-related toxicity and side effects, and further help improve patient compliance.

### Example 2. Tumor Volume Reduction and Body Weight Change in Mice During Combination Therapy in a PDX Model with Patient LU-01-0030 FP10

The study was conducted in xenograft tumor PDX model established with surgical tumor tissues from KRAS^{G12C} NSCLC patient LU-01-0030 FP10. Briefly, 6-8 week-old female Balb/c nude mice were used for the studies. Tumor samples obtained from patients were immediately transferred into tissue preservation solution and sliced into small fragments. Mice were inoculated with the fragments subcutaneously at one flank to produce xenografts called passage 1 (P1). The serial xenografts of different passages were generated using the same procedure. A tumor tissue size of 20˜30 mm³ was implanted into Balb/c nude mice. When the average tumor size reached approximately ˜166 mm³ in the mice, the animals were randomly allocated into different groups, with 3 mice per group. The day of randomization was defined as study day 0. Tumor volume is expressed in mm³ using the following formula: V (volume)=(a×b²)/2 where a and b are the long and short diameters of the tumor, respectively. RTV is the relative tumor volume, defined as RTV=Vt/V₀, where V₀ is the tumor volume at study start (D₀), Vt is the tumor volume at time of measurement (i.e., same time for both treatment and vehicle control group). The relative tumor growth rate T/C is calculated as T/C %=T_{RTV}/C_{RTV}×100% (T_{RTV}: treatment group RTV: C_{RTV}: vehicle control group RTV). The tumor growth inhibition (TGI %) is calculated as TGI (%)=[1−(average tumor volume of treatment group at study completion −average tumor volume of treatment group at study initiation)/(average tumor volume of vehicle control group at study completion−average tumor volume of vehicle control group at study initiation)]*100.

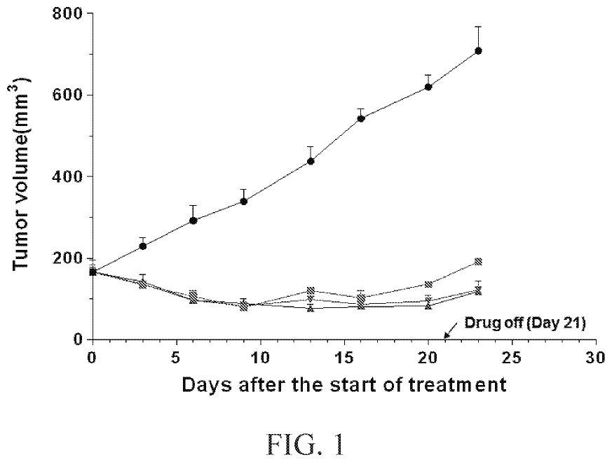

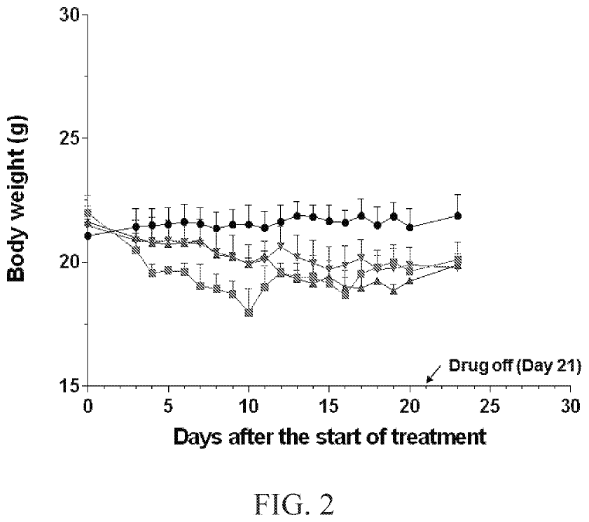

Mean tumor volumes during the combination therapy are shown in FIG. 1. Mean body weights of mice are shown in FIG. 2. Mean tumor volumes are also shown below in Table 5A. The data presented in FIG. 1 shows significant tumor growth inhibition in the KRAS^{G12C} mutant NSCLC model (see also Tables 7A and 7C). The data presented in FIG. 2 shows the combination therapy is tolerable in animals, especially at dosage levels which showed clear in vivo efficacy.

TABLE 7A
Mean Tumor Volume (mm³) during Combination Therapy (PDX Model with Patient LU-01-0030 FP10)

| | Study Days | | | | | | | | |
|---|---|---|---|---|---|---|---|---|---|
| Samples | 0 | 3 | 6 | 9 | 13 | 16 | 20 | 23 | 27 |
| Control | 166 ± 11 | 229 ± 22 | 292 ± 37 | 339 ± 29 | 437 ± 37 | 542 ± 25 | 620 ± 29 | 709 ± 60 | 738 ± 32 |
| Group 1 | 167 ± 17 | 134 ± 9 | 106 ± 14 | 79 ± 3 | 121 ± 7 | 104 ± 17 | 136 ± 7 | 191 ± 9 | 329 ± 42 |
| osimertinib 10 mg/kg, | | | | | | | | | |
| cobimetinib 5 mg/kg, | | | | | | | | | |
| palbociclib 20 mg/kg | | | | | | | | | |
| Group 2 | 166 ± 12 | 142 ± 18 | 106 ± 14 | 89 ± 13 | 77 ± 11 | 82 ± 15 | 83 ± 14 | 119 ± 10 | 156 ± 25 |
| cetuximab 1 mg/kg, | | | | | | | | | |
| cobimetinib 5 mg/kg | | | | | | | | | |
| palbociclib 20 mg/kg | | | | | | | | | |
| Group 3 | 168 ± 28 | 137 ± 23 | 97 ± 20 | 84 ± 9 | 98 ± 16 | 88 ± 14 | 96 ± 13 | 122 ± 22 | 222 ± 44 |
| avitinib 100 mg/kg, | | | | | | | | | |
| cobimetinib 5 mg/kg, | | | | | | | | | |
| palbociclib 20 mg/kg | | | | | | | | | |

TABLE 7B
Study Design for Combination Therapy (PDX
Model with Patient LU-01-0030 FP10)

| Group | Study Design |
|---|---|
| Control | Vehicle (no treatment) |
| 1 | osimertinib 10 mg/kg, cobimetinib 5 mg/kg, and palbociclib 20 |
| | mg/kg daily (p.o.) for 21 days, then no treatment for 7 days |
| 2 | cetuximab 1 mg/kg per week (i.p.), cobimetinib 5 mg/kg and |
| | palbociclib 20 mg/kg daily (p.o.) for 21 days, then no |
| | treatment for 7 days |
| 3 | avitinib 100 mg/kg, cobimetinib 5 mg/kg, and palbociclib 20 |
| | mg/kg daily (p.o.) for 21 days, then no treatment for 7 days |

TABLE 7C
Tumor Growth Inhibition in NSCLC PDX Model from
Patient no. LU-01-0030 with KRAS G12C Mutation

| | Tumor Volume | | | |
|---|---|---|---|---|
| | (mm³)^{a} | T/C^{b} | TGI^{b} | |
| Group | (Day 27) | (%) | (%) | P value^{c} |
| Control (Vehicle control) | 738 ± 32 | — | — | — |
| Group 1 | 329 ± 42 | 44.2 | 71.7 | <0.001 |
| Group 2 | 156 ± 25 | 20.9 | 101.7 | <0.001 |
| Group 3 | 222 ± 44 | 29.3 | 90.6 | <0.001 |

Note:
^{a}average ± standard deviation
^{b}Tumor growth inhibition parameters T/C and TGI (TGI %) = [1 − (T₂₇ − T₀)/(V₂₇ − V₀)] × 100)
^{c}p value calculated based on T/C %

### Example 3. Tumor Volume Reduction and Body Weight Change in Mice During Combination Therapy in a PDX Model with Patient LU6419 FP10

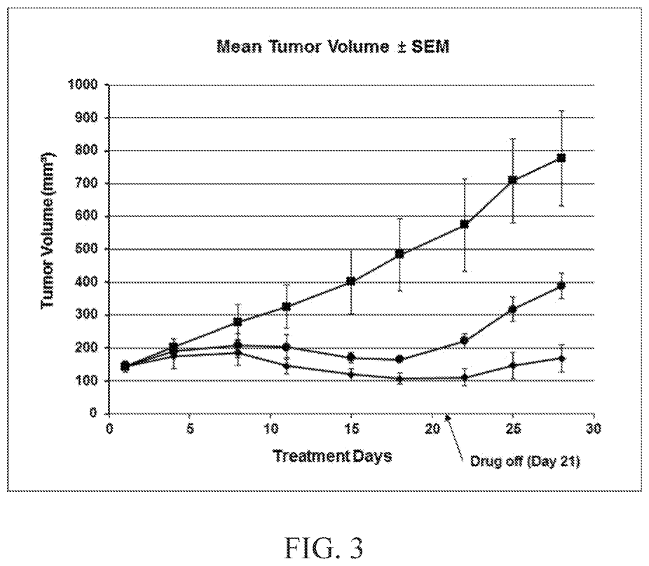

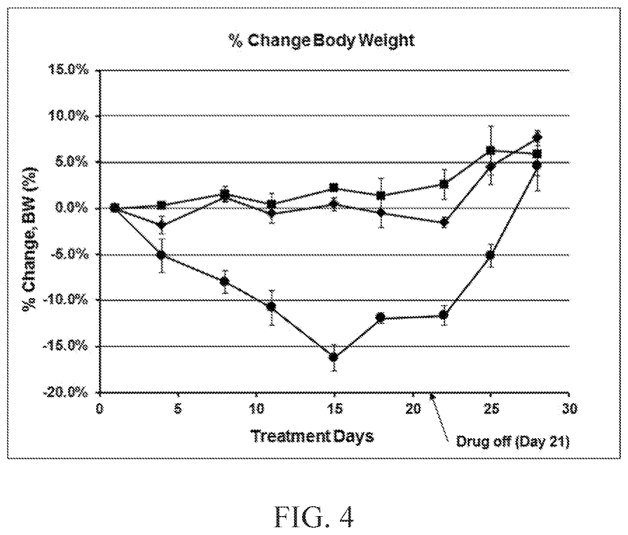

The same procedures were followed as in Example 2 with KRAS^{G12V} NSCLC patient LU6419 FP10 using various combinations. Tumor volume values and body weight changes are shown in FIGS. 3 and 4, respectively. Mean tumor volumes are also shown below in Table 8A. Both combinations: osimertinib+cobimetinib+palbociclib and cetuximab+cobimetinib+palbociclib showed significant tumor growth inhibition (Tables 8A and 8C) and showed tolerability in this model.

TABLE 8A
Mean Tumor Volume (mm³) during Combination Therapy (PDX Model with Patient LU6419 FP10)

| | Study Days | | | | |
|---|---|---|---|---|---|
| Samples | 1 | 4 | 8 | 11 | 15 |
| Control | 142.84 ± 17.14 | 203.17 ± 25.05 | 277.41 ± 52.80 | 324.19 ± 66.45 | 401.10 ± 98.72 |
| Group 1 | 143.70 ± 17.36 | 175.26 ± 36.84 | 185.37 ± 36.56 | 145.67 ± 24.37 | 119.44 ± 18.45 |
| cetuximab 1 mg/kg, | | | | | |
| cobimetinib 5 mg/kg | | | | | |
| palbociclib 20 mg/kg | | | | | |
| Group 2 | 142.82 ± 14.82 | 189.18 ± 11.44 | 207.58 ± 36.25 | 202.97 ± 38.06 | 170.07 ± 15.60 |
| osimertinib 10 mg/kg, | | | | | |
| cobimetinib 5 mg/kg, | | | | | |
| palbociclib 20 mg/kg | | | | | |

| | Study Days | | | |
|---|---|---|---|---|
| Samples | 18 | 22 | 25 | 28 |
| Control | 483.26 ± 109.53 | 573.63 ± 141.91 | 708.90 ± 127.66 | 776.94 ± 145.39 |
| Group 1 | 106.84 ± 16.89 | 111.12 ± 24.96 | 146.80 ± 39.52 | 168.34 ± 42.68 |
| cetuximab 1 mg/kg, | | | | |
| cobimetinib 5 mg/kg | | | | |
| palbociclib 20 mg/kg | | | | |
| Group 2 | 164.86 ± 7.12 | 221.47 ± 21.14 | 317.04 ± 36.82 | 388.03 ± 39.72 |
| osimertinib 10 mg/kg, | | | | |
| cobimetinib 5 mg/kg, | | | | |
| palbociclib 20 mg/kg | | | | |

TABLE 8B
Study Design for Combination Therapy
(PDX Model with Patient LU6419 FP10)

| Group | Study Design |
|---|---|
| Control | Vehicle (no treatment) |
| 1 | cetuximab 1 mg/kg per week (i.p.), cobimetinib 5 mg/kg and |
| | palbociclib 20 mg/kg daily (p.o.) for 21 days, then no |
| | treatment for 7 days |
| 2 | osimertinib 10 mg/kg, cobimetinib 5 mg/kg, and palbociclib 20 |
| | mg/kg daily (p.o.) for 21 days, then no treatment for 7 days |

TABLE 8C
Tumor Growth Inhibition Parameters (PDX
Model with Patient LU6419 FP10)

| | Tumor volume | Tumor volume | | |
|---|---|---|---|---|
| | (mm³) ^{a} | (mm³) ^{a} | | |
| Group | day 1 | day 28 | TGI % ^{b} | p Value ^{c} |
| Control | 142.83 +/− | 776.94 +/− | — | — |
| | 17.14(3) | 145.39(3) | | |
| Group 1 | 143.70 +/− | 168.34 +/− | 96.11% | 0.00561 |
| | 17.36(3) | 42.68(3) | | |
| Group 2 | 142.82 +/− | 388.03 +/− | 61.33% | 0.0399 |
| | 14.82(3) | 39.72(3) | | |

Note:
^{a} average ± standard deviation (number of animals)
^{b} Tumor growth inhibition parameters TGI (TGI %) = [1 − (T₂₈ − T₁)/(V₂₈ − V₁)] × 100), where T₁ and T₂₈ is the tumor volume at Day 1 and Day 28 respectively.
^{c} p value calculated based on T/C %

### Example 4. Tumor Volume Reduction and Body Weight Change in Mice During Combination Therapy in a PDX Model with Patient LU2071

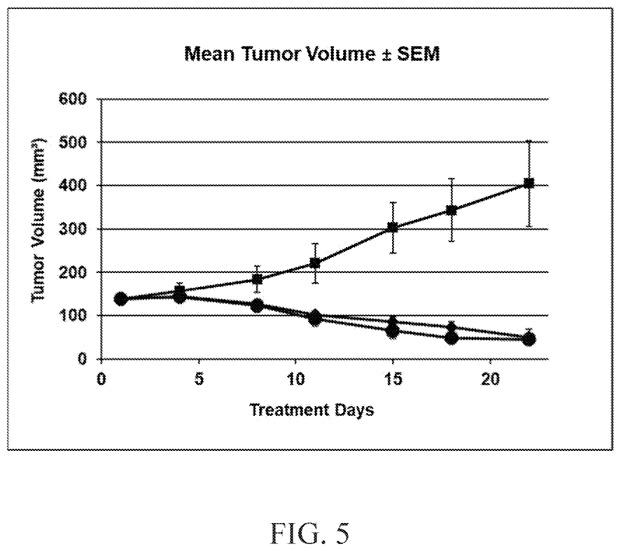

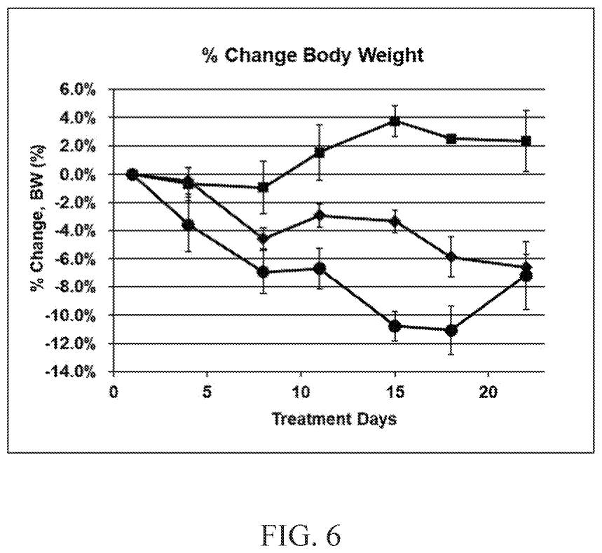

The same procedures were followed as in Example 2 with KRAS^{G12V} NSCLC patient LU2071 using various combinations. Tumor volume values and body weight changes are shown in FIGS. 5 and 6, respectively. Mean tumor volumes are also shown below in Table 9A. Both combinations: osimertinib+cobimetinib+palbociclib and cetuximab+cobimetinib+palbociclib significantly reduced tumor volumes and caused tumor regression (Tables 9A and 9C). Both combinations showed good tolerability in this model.

TABLE 9A
Mean Tumor Volume (mm³) during Combination Therapy (PDX Model with Patient LU2071)

| | Study Days | | | | |
|---|---|---|---|---|---|
| Samples | 1 | 4 | 8 | 11 | 15 |
| Control | 138.33 ± 9.78 | 157.02 ± 18.56 | 183.68 ± 30.48 | 221.57 ± 45.36 | 303.20 ± 58.28 |
| Group 1 | 137.64 ± 11.07 | 143.95 ± 13.42 | 126.45 ± 11.46 | 102.36 ± 7.66 | 85.28 ± 11.41 |
| cetuximab 1 mg/kg, | | | | | |
| cobimetinib 5 mg/kg | | | | | |
| palbociclib 20 mg/kg | | | | | |
| Group 2 | 139.33 ± 10.80 | 142.62 ± 10.79 | 123.07 ± 10.97 | 92.65 ± 17.74 | 65.34 ± 18.09 |
| osimertinib 10 mg/kg, | | | | | |
| cobimetinib 5 mg/kg, | | | | | |
| palbociclib 20 mg/kg | | | | | |

| | Study Days | | | |
|---|---|---|---|---|
| Samples | 18 | 22 | 25 | 29 |
| Control | 343.04 ± 72.43 | 405.29 ± 98.67 | 448.91 ± 106.27 | 478.31 ± 106.46 |
| Group 1 | 73.09 ± 12.24 | 50.61 ± 17.95 | 45.29 ± 15.80 | 38.42 ± 13.98 |
| cetuximab 1 mg/kg, | | | | |
| cobimetinib 5 mg/kg | | | | |
| palbociclib 20 mg/kg | | | | |
| Group 2 | 48.42 ± 11.48 | 45.49 ± 8.83 | 37.49 ± 10.20 | 33.70 ± 11.26 |
| osimertinib 10 mg/kg, | | | | |
| cobimetinib 5 mg/kg, | | | | |
| palbociclib 20 mg/kg | | | | |

TABLE 9B
Study Design for Combination Therapy
(PDX Model with Patient LU2071)

| Group | Study Design |
|---|---|
| Control | Vehicle (no treatment) |
| 1 | cetuximab 1 mg/kg per week (i.p.), cobimetinib 5 mg/kg and |
| | palbociclib 20 mg/kg daily (p.o.) for 21 days, then no |
| | treatment for 7 days |
| 2 | osimertinib 10 mg/kg, cobimetinib 5 mg/kg, and palbociclib 20 |
| | mg/kg daily (p.o.) for 21 days, then no treatment for 7 days |

TABLE 9C
Tumor Growth Inhibition Parameters
(PDX Model with Patient LU2071)

| | Tumor volume | Tumor volume | | | |
|---|---|---|---|---|---|
| | (mm³) ^{a} | (mm³) ^{a} | TGI | | p |
| Group | day 1 | day 28 | % ^{b} | REG ^{c} | Value ^{d} |
| Control | 138.33 +/− | 1120.56 +/− | — | — | — |
| | 9.78(3) | 131.03(3) | | | |
| Group 1 | 137.64 +/− | 10.77 +/− | 112.92% | 92.17% | 0.042 |
| | 11.08(3) | 7.02(3) | | | |
| Group 2 | 139.33 +/− | 3.82 +/− | 113.80% | 92.17% | 0.017 |
| | 10.80(3) | 3.82(3) | | | |

Note:
^{a} average ± standard deviation (number of animals)
^{b} Tumor growth inhibition parameter TGI (TGI %) = [1 − (T₂₈ − T₁)/(V₂₈ − V₁)] × 100), where T₁ and T₂₈ is the tumor volume at Day 1 and Day 28 respectively.
^{c} Tumor regression parameter is calculated as % REG = [(T₁ − T₂₈)/T1] × 100%, where T₁ and T₂₈ is the tumor volume at Day 1 and Day 28 respectively.
^{d} p value calculated based on T/C %

### Example 5. Tumor Volume Reduction and Body Weight Change in Mice During Combination Therapy in a PDX Model with Patient LU6405

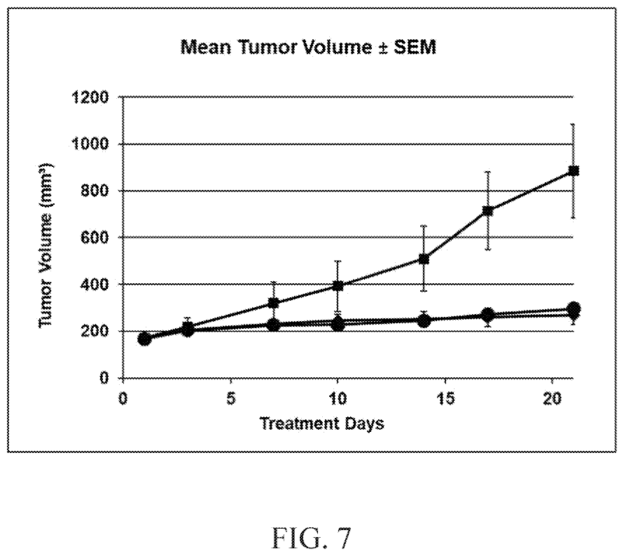

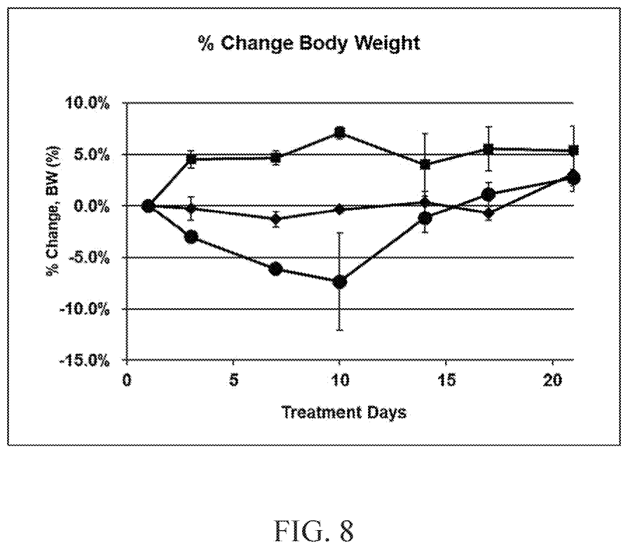

The same procedures were followed as in Example 2 with KRAS^{G12C} NSCLC patient LU6419 FP10 using various combinations. Tumor volume values and body weight changes are shown in FIGS. 7 and 8, respectively. Mean tumor volumes are also shown below in Table 10A. Both combinations: osimertinib+cobimetinib+palbociclib and cetuximab+cobimetinib+palbociclib caused significant tumor growth inhibition (Table 10A and 10C) and showed tolerability in this model.

TABLE 10A
Mean Tumor Volume (mm³) during Combination Therapy (PDX Model with Patient LU6405)

| | Study Days | | | | |
|---|---|---|---|---|---|
| Samples | 1 | 3 | 7 | 10 | 14 |
| Control | 170.35 ± 13.91 | 219.10 ± 38.17 | 319.51 ± 89.46 | 392.61 ± 107.50 | 509.82 ± 137.71 |
| Group 1 | 168.98 ± 8.78 | 205.46 ± 17.20 | 231.02 ± 21.80 | 245.97 ± 26.54 | 253.19 ± 30.28 |
| cetuximab 1 mg/kg, | | | | | |
| cobimetinib 5 mg/kg | | | | | |
| palbociclib 20 mg/kg | | | | | |
| Group 2 | 167.70 ± 7.36 | 203.75 ± 21.45 | 226.38 ± 26.79 | 228.99 ± 17.88 | 245.77 ± 7.97 |
| osimertinib 10 mg/kg, | | | | | |
| cobimetinib 5 mg/kg, | | | | | |
| palbociclib 20 mg/kg | | | | | |

| | Study Days | | | |
|---|---|---|---|---|
| Samples | 17 | 21 | 23 | 27 |
| Control | 714.81 ± 166.23 | 883.96 ± 201.08 | 1053.80 ± 248.71 | 1234.73 ± 305.44 |
| Group 1 | 259.70 ± 39.10 | 268.39 ± 40.06 | 285.46 ± 42.17 | 318.16 ± 36.67 |
| cetuximab 1 mg/kg, | | | | |
| cobimetinib 5 mg/kg | | | | |
| palbociclib 20 mg/kg | | | | |
| Group 2 | 271.51 ± 14.33 | 296.35 ± 20.84 | 311.25 ± 24.02 | 356.39 ± 11.46 |
| osimertinib 10 mg/kg, | | | | |
| cobimetinib 5 mg/kg, | | | | |
| palbociclib 20 mg/kg | | | | |

TABLE 10B
Study Design for Combination Therapy
(PDX Model with Patient LU6405)

| Group | Study Design |
|---|---|
| Control | Vehicle (no treatment) |
| 1 | cetuximab 1 mg/kg per week (i.p.), cobimetinib 5 mg/kg and |
| | palbociclib 20 mg/kg daily (p.o.) for 21 days, then no treatment |
| | for 7 days |
| 2 | osimertinib 10 mg/kg, cobimetinib 5 mg/kg, and palbociclib 20 |
| | mg/kg daily (p.o.) for 21 days, then no treatment for 7 days |

TABLE 10C
Tumor Growth Inhibition Parameters
(PDX Model with Patient LU6405)

| | Tumor volume | Tumor volume | | |
|---|---|---|---|---|
| | (mm³) ^{a} | (mm³) ^{a} | | |
| Group | day 1 | day 28 | TGI % ^{b} | p Value ^{c} |
| Control | 170.34 +/− | 2350.58 +/− | — | — |
| | 13.91(3) | 236.26(3) | | |
| Group 1 | 168.98 +/− | 413.79 +/− | 88.77% | 0.00059 |
| | 8.79(3) | 24.30(3) | | |
| Group 2 | 167.70 +/− | 505.45 +/− | 84.51% | 0.018 |
| | 7.36(3) | 19.74(3) | | |

Note:
^{a} average ± standard deviation (number of animals)
^{b} Tumor growth inhibition parameters TGI (TGI %) = [1 − (T₂₈ −T₁)/(V₂₈ − V₁)] × 100), where T₁ and T₂₈ is the tumor volume at Day 1 and Day 28 respectively.
^{c} p value calculated based on T/C %

### Example 6. Tumor Volume Reduction and Body Weight Change in Mice During Combination Therapy in a CDX Model with H358 (KRAS^{G12C} NSCLC)

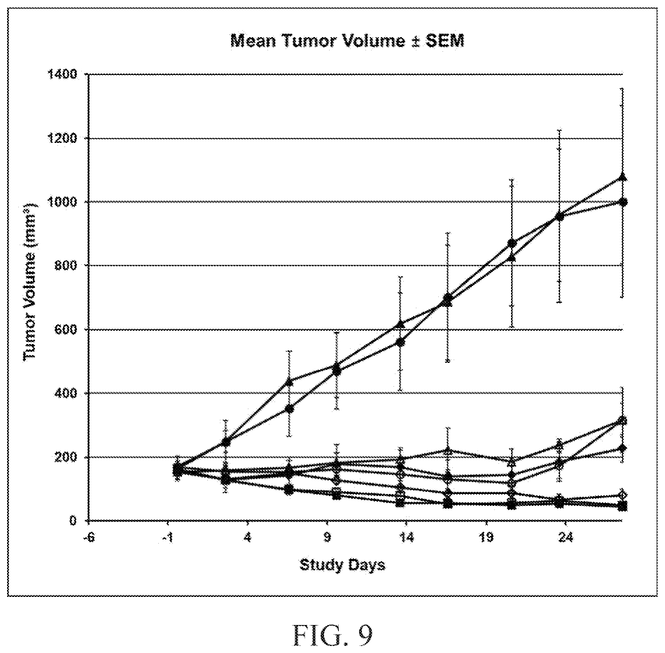

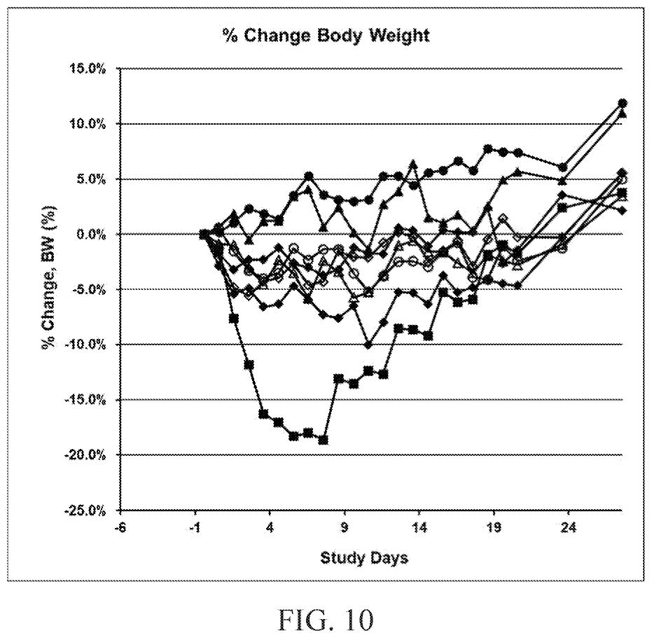

The same procedures were followed as in Example 1 with H358 cell lines. Tumor volume values and body weight changes are shown in FIGS. 9 and 10 respectively. Mean tumor volumes are also shown below in Table 11A. All triplet combinations: osimertinib+cobimetinib+palbociclib, cetuximab+cobimetinib+palbociclib, cetuximab+TAK-733+ palbociclib significantly inhibited tumor growth (Table 11A and 11C) and showed tolerability in this model. In particular, triplet combinations of cetuximab 1 mg/kg+cobimetinib 5 mg/kg+palbociclib 20 mg/kg (Group 7), cetuximab 1 mg/kg+TAK-733 10 mg/kg+palbociclib 20 mg/kg (Group 2), and cetuximab 1 mg/kg+TAK-733 30 mg/kg+palbociclib 20 mg/kg (Group 3) caused tumor regression. Furthermore, all triplet combinations showed better tumor growth inhibition than doublet combinations of TAK-733 10 mg/kg+palbociclib 20 mg/kg; and cobimetinib 5 mg/kg+palbociclib 20 mg/kg. The chemotherapeutic agent cisplatin at 4 mg/kg showed no effect on inhibiting tumor growth.

TABLE 11A
Mean Tumor Volume (mm³) during Combination Therapy (CDX Model with H358)

| | Study Days | | | | |
|---|---|---|---|---|---|
| Samples | 0 | 3 | 7 | 10 | 14 |
| Control | 170.65 ± 29.11 | 247.47 ± 67.55 | 352.49 ± 87.55 | 469.79 ± 119.05 | 561.62 ± 152.03 |
| Group 1 | 163.26 ± 18.67 | 130.14 ± 41.58 | 144.10 ± 56.42 | 179.08 ± 60.43 | 169.66 ± 60.13 |
| osimertinib 10 mg/kg, | | | | | |
| cobimetinib 5 mg/kg, | | | | | |
| palbociclib 20 mg/kg | | | | | |
| Group 2 | 164.80 ± 24.16 | 128.55 ± 24.95 | 99.61 ± 17.17 | 90.55 ± 9.57 | 78.52 ± 10.60 |
| cetuximab 1 mg/kg, | | | | | |
| TAK-733 10 mg/kg | | | | | |
| palbociclib 20 mg/kg | | | | | |
| Group 3 | 161.11 ± 36.04 | 130.69 ± 17.27 | 98.04 ± 4.53 | 80.42 ± 9.60 | 56.82 ± 8.83 |
| cetuximab 1 mg/kg, | | | | | |
| TAK-733 30 mg/kg (3 | | | | | |
| mg/kg after day 10) | | | | | |
| palbociclib 20 mg/kg | | | | | |
| Group 4 | 154.03 ± 23.75 | 159.80 ± 25.62 | 167.82 ± 22.27 | 183.68 ± 29.66 | 193.76 ± 29.40 |
| cobimetinib 5 mg/kg, | | | | | |
| palbociclib 20 mg/kg | | | | | |
| Group 5 | 168.51 ± 34.63 | 154.79 ± 19.70 | 153.21 ± 16.92 | 162.96 ± 20.08 | 146.85 ± 32.72 |
| TAK-733 10 mg/kg | | | | | |
| palbociclib 20 mg/kg | | | | | |
| Group 6 | 164.52 ± 22.82 | 249.59 ± 33.58 | 438.18 ± 93.81 | 489.05 ± 102.01 | 618.71 ± 145.67 |
| cisplatin 4 mg/kg | | | | | |
| Group 7 | 153.29 ± 11.01 | 131.80 ± 7.50 | 150.94 ± 5.85 | 127.80 ± 11.39 | 106.70 ± 19.40 |
| cetuximab 1 mg/kg, | | | | | |
| cobimetinib 5 mg/kg | | | | | |
| palbociclib 20 mg/kg | | | | | |

| | Study Days | | | |
|---|---|---|---|---|
| Samples | 17 | 21 | 24 | 28 |
| Control | 701.05 ± 202.08 | 871.65 ± 196.54 | 954.72 ± 270.00 | 1000.90 ± 300.49 |
| Group 1 | 140.65 ± 50.27 | 144.98 ± 51.16 | 186.15 ± 61.11 | 227.64 ± 42.57 |
| osimertinib 10 mg/kg, | | | | |
| cobimetinib 5 mg/kg, | | | | |
| palbociclib 20 mg/kg | | | | |
| Group 2 | 53.52 ± 9.82 | 56.54 ± 14.67 | 62.56 ± 14.02 | 49.00 ± 6.60 |
| cetuximab 1 mg/kg, | | | | |
| TAK-733 10 mg/kg | | | | |
| palbociclib 20 mg/kg | | | | |
| Group 3 | 55.42 ± 11.03 | 49.92 ± 11.33 | 54.32 ± 13.93 | 44.37 ± 13.66 |
| cetuximab 1 mg/kg, | | | | |
| TAK-733 30 mg/kg (3 | | | | |
| mg/kg after day 10) | | | | |
| palbociclib 20 mg/kg | | | | |
| Group 4 | 222.12 ± 69.00 | 185.83 ± 40.60 | 238.55 ± 18.92 | 316.08 ± 51.71 |
| cobimetinib 5 mg/kg, | | | | |
| palbociclib 20 mg/kg | | | | |
| Group 5 | 131.18 ± 29.78 | 118.77 ± 24.30 | 172.82 ± 40.33 | 316.40 ± 101.56 |
| TAK-733 10 mg/kg | | | | |
| palbociclib 20 mg/kg | | | | |
| Group 6 | 685.80 ± 180.07 | 828.32 ± 220.11 | 958.50 ± 207.93 | 1079.33 ± 275.10 |
| cisplatin 4 mg/kg | | | | |
| Group 7 | 86.84 ± 14.59 | 87.68 ± 24.64 | 66.91 ± 16.16 | 80.56 ± 20.52 |
| cetuximab 1 mg/kg, | | | | |
| cobimetinib 5 mg/kg | | | | |
| palbociclib 20 mg/kg | | | | |

TABLE 11B
Study Design for Combination Therapy (CDX Model with H358)

| Group | Study Design |
|---|---|
| Control | Vehicle (no treatment) |
| 1 | osimertinib 10 mg/kg, cobimetinib 5 mg/kg, and palbociclib 20 |
| | mg/kg daily (p.o.) for 21 days, then no treatment for 7 days |
| 2 | cetuximab 1 mg/kg per week (i.p.), TAK-733 30 mg/kg and |
| | palbociclib 20 mg/kg daily (p.o.) for 21 days, then no |
| | treatment for 7 days |
| 3 | cetuximab 1 mg/kg per week (i.p.), TAK-733 30 mg/kg and |
| | palbociclib 20 mg/kg daily (p.o.) for 21 days; after Day 10, |
| | TAK-733 reduced to 3 mg/kg, then no treatment for 7 days |
| 4 | cobimetinib 5 mg/kg and palbociclib 20 mg/kg daily (p.o.) for |
| | 21 days, then no treatment for 7 days |
| 5 | TAK-733 10 mg/kg and palbociclib 20 mg/kg daily (p.o.) for |
| | 21 days, then no treatment for 7 days |
| 6 | cisplatin 4 mg/kg per week (i.p.) for 21 days, then no treatment |
| | for 7 days |
| 7 | cetuximab 1 mg/kg per week (i.p.), cobimetinib 5 mg/kg and |
| | palbociclib 20 mg/kg daily (p.o.) or 21 days, then no treatment |
| | for 7 days |

TABLE 11C
Tumor Growth Inhibition Parameters (CDX Model with H358)

| | Tumor volume | Tumor volume | | |
|---|---|---|---|---|
| | (mm³) ^{a} | (mm³) ^{a} | | |
| Group | day 0 | day 28 | TGI % ^{b} | REG ^{c} |
| Control | 170.65 ± 29.11 | 1000.90 ± 300.49 | — | — |
| Group 1 | 163.26 ± 18.67 | 227.64 ± 42.57 | 92.2% | — |
| Group 2 | 164.80 ± 24.16 | 49.00 ± 6.60 | 113.9% | 70.3% |
| Group 3 | 161.11 ± 36.04 | 44.37 ± 13.66 | 114.1% | 72.5% |
| Group 4 | 154.03 ± 23.75 | 316.08 ± 51.71 | 80.5% | — |
| Group 5 | 168.51 ± 34.63 | 316.40 ± 101.56 | 82.2% | — |
| Group 6 | 164.52 ± 22.82 | 1079.33 ± 275.10 | −10.2% | — |
| Group 7 | 153.29 ± 11.01 | 80.56 ± 20.52 | 108.8% | 47.4% |

Note:
^{a} average ± standard deviation
^{b} Tumor growth inhibition parameter TGI (TGI %) = [1 − (T₂₈ − T₀)/(V₂₈ − V₀)] × 100), where T₀ and T₂₈ is the tumor volume at Day 0 and Day 28 respectively.
^{c} Tumor regression parameter is calculated as % REG = [(T₀ − T₂₈)/T₀] × 100%, where T₀ and T₂₈ is the tumor volume at Day 0 and Day 28 respectively.

1. A composition comprising:
(a) an epidermal growth factor receptor (EGFR) inhibitor;
(b) a mitogen-activated protein kinase (MEK) 1/2 inhibitor; and
(c) a cyclin dependent kinase (CDK) 4/6 inhibitor;
wherein the composition does not comprises a KRAS inhibitor.

2.-5. (canceled)

6. A method of treating or delaying progression of cancer in a subject comprising administering to the subject an affective amount of
(a) an epidermal growth factor receptor (EGFR) inhibitor;
(b) a mitogen-activated protein kinase (MEK) 1/2 inhibitor; and
(c) a cyclin dependent kinase (CDK) 4/6 inhibitor;
wherein the subject has cancer that has a KRAS mutation or is at risk of developing cancer that has a KRAS mutation.

7. The method of claim 6, wherein a KRAS inhibitor is not administered to the subject.

8. The method of claim 6, wherein the EGFR inhibitor is osimertinib or a salt thereof, avitinib or a salt thereof, or cetuximab, wherein the MEK 1/2 inhibitor is cobimetinib or a salt thereof, trametinib or a salt thereof, binimetinib or a salt thereof, or TAK-733 or a salt thereof, and wherein the CDK 4/6 inhibitor is palbociclib or a salt thereof, or abemaciclib or a salt thereof.

9. The method of claim 8, wherein the method comprises administering an affective amount of osimertinib or a salt thereof, cobimetinib or a salt thereof, and palbociclib or a salt thereof.

10. The method of claim 8, wherein the method comprises administering to the subject an effective amount of osimertinib or a salt thereof, TAK-733 or a salt thereof, and palbociclib or a salt thereof.

11. The method of claim 8, wherein the method comprises administering to the subject an effective amount of osimertinib or a salt thereof, trametinib or a salt thereof, and palbociclib or a salt thereof.

12. The method of claim 8, wherein the method comprises administering to the subject an affective amount of cetuximab, cobimetinib or a salt thereof, and palbociclib or a salt thereof.

13. The method of claim 8, wherein the method comprises administering to the subject an affective amount of cetuximab, TAK-733 or a salt thereof, and palbociclib or a salt thereof.

14. The method of claim 8, wherein the method comprises administering to the subject an effective amount of cetuximab, trametinib or a salt thereof, and palbociclib or a salt thereof.

15. The method of claim 8, wherein the method comprises administering to the subject an effective amount of osimertinib or a salt thereof, binimetinib or a salt thereof, and palbociclib or a salt thereof.

16. The method of claim 8, wherein the method comprises administering to the subject an effective amount of cetuximab, binimetinib or a salt thereof, and palbociclib or a salt thereof.

17. The method of claim 8, wherein the method comprises administering to the subject an effective amount of avitinib or a salt thereof, cobimetinib or a salt thereof, and palbociclib or a salt thereof.

18. The method of claim 8, wherein the method comprises administering to the subject an affective amount of cetuximab, cobimetinib or a salt thereof, and abemaciclib or a salt thereof.

19. The method of claim 9, wherein osimertinib or a salt thereof, cobimetinib or a salt thereof, and palbociclib or a salt thereof are administered in one composition, administered simultaneously to the subject, or administered intermittently to the subject.

20. (canceled)

21. (canceled)

22. The method of claim 12, wherein cetuximab, cobimetinib or a salt thereof, and palbociclib or a salt thereof are administered in two or three compositions or separately.

23. The method of claim 13, wherein cetuximab, TAK-733 or a salt thereof, and palbociclib or a salt thereof are administered in two or three compositions or separately.

24. The method of claim 6, wherein the EGFR inhibitor, the MEK 1/2 inhibitor, and the CDK 4/6 inhibitor are administered in one composition or two or three compositions, administered simultaneously to the subject, administered separately to the subject, or administered intermittently to the subject.

25. The method of claim 6, wherein the cancer has a KRAS G12 mutation or a KRAS G13 mutation.

26. The method of claim 25, wherein the KRAS G12 mutation is G12C, G12V, or G12D mutation, and/or wherein the KRAS G13 mutation is G13D mutation.

27. The method of claim 6, wherein the cancer is a malignant epithelial tumor or carcinoma.

28. The method of claim 27, wherein the cancer is a carcinoma selected from the group consisting of a lung cancer and pancreatic cancer.

29. The method of claim 6, wherein the cancer is a lung cancer.

30. The method of claim 29, wherein the lung cancer is non-small cell lung cancer (NSCLC).

31. The method of claim 30, wherein the NSCLC has a KRAS G12C, G12D, or G12V mutation and/or KRAS G13D mutation.

32. The method of claim 6, wherein the method reduces cancer cell growth and/or increase cancer cell-killing by about 20-99% more than administration of (a) the EGFR inhibitor, (b) the MEK 1/2 inhibitor or (c) the CDK 4/6 inhibitor alone.

33. The method of claim 6, wherein the method reduces mean tumor volume by about 20-95%.

34. The method of claim 9, wherein osimertinib or a salt thereof is administered to the subject in a daily dose of about 40-80 mg.

35. The method of claim 9, wherein cobimetinib or a salt thereof is administered to the subject in a daily dose of about 20-60 mg.

36. The method of claim 9, wherein palbociclib or a salt thereof is administered to the subject in a daily dose of about 75-125 mg.

37. The method of claim 12, wherein cetuximab is administered to the subject in a weekly dose of about 400 mg/m² infused over 120 minutes with a maximum infusion rate of 10 mg/min, followed by weekly dose of 250 mg/m² infused over 60 minutes with a maximum infusion rate of 10 mg/min.

38. The method of claim 6, wherein the subject is a human.

39. The method of claim 6, wherein the method reduces the tumor volume by at least about 85%.

40. A kit comprising:
(a) an epidermal growth factor receptor (EGFR) inhibitor;
(b) a mitogen-activated protein kinase (MEK) 1/2 inhibitor; and
(c) a cyclin dependent kinase (CDK) 4/6 inhibitor;
wherein the kit does not comprises a KRAS inhibitor.

41.-63. (canceled)
